SEQUENCE LISTING (49 sequences)

SEQ ID NO: 1 - DNA, 32 bp, synthetic construct
  source 1..32: mol_type = other DNA
gcctgttgggctagctacaacgacagatattt

SEQ ID NO: 2 - DNA, 33 bp, synthetic construct
  source 1..33: mol_type = other DNA
gttgtcagaggctagctacaacgaatttttccg

SEQ ID NO: 3 - DNA, 33 bp, synthetic construct
  source 1..33: mol_type = other DNA
ccagatccaggctagctacaacgattctatcat

SEQ ID NO: 4 - DNA, 32 bp, synthetic construct
  source 1..32: mol_type = other DNA
tagagtaagggctagctacaacgaagttcgcc

SEQ ID NO: 5 - DNA, 33 bp, synthetic construct
  source 1..33: mol_type = other DNA
ttttagttgggctagctacaacgagttagtact

SEQ ID NO: 6 - DNA, 35 bp, synthetic construct
  source 1..35: mol_type = other DNA
tattttagcaggctagctacaacgaagtcatttaa

SEQ ID NO: 7 - DNA, 35 bp, synthetic construct
  source 1..35: mol_type = other DNA
tgactcataaggctagctacaacgatattttagca

SEQ ID NO: 8 - DNA, 37 bp, synthetic construct
  source 1..37: mol_type = other DNA
tgactcataatggctagctacaacgatttagcatagt

SEQ ID NO: 9 - DNA, 33 bp, synthetic construct
  source 1..33: mol_type = other DNA
gcttttttaggctagctacaacgatgactaatg

SEQ ID NO: 10 - DNA, 35 bp, synthetic construct
  source 1..35: mol_type = other DNA
agcttttttaggctagctacaacgatgactaatgg

SEQ ID NO: 11 - DNA, 33 bp, synthetic construct
  source 1..33: mol_type = other DNA
ttagcatagggctagctacaacgacatttaaat

SEQ ID NO: 12 - DNA, 37 bp, synthetic construct
  source 1..37: mol_type = other DNA
ttttagcatagggctagctacaacgacatttaaataa

SEQ ID NO: 13 - DNA, 33 bp, synthetic construct
  source 1..33: mol_type = other DNA
attttagcaggctagctacaacgaagtcattta

SEQ ID NO: 14 - DNA, 37 bp, synthetic construct
  source 1..37: mol_type = other DNA
ttattttagcaggctagctacaacgaagtcatttaaa

SEQ ID NO: 15 - DNA, 33 bp, synthetic construct
  source 1..33: mol_type = other DNA
gactcataaggctagctacaacgatattttagc

SEQ ID NO: 16 - DNA, 33 bp, synthetic construct
  source 1..33: mol_type = other DNA
tttagtaagggctagctacaacgatctttatgg

SEQ ID NO: 17 - DNA, 35 bp, synthetic construct
  source 1..35: mol_type = other DNA
atttagtaagggctagctacaacgattatgggtac

SEQ ID NO: 18 - DNA, 33 bp, synthetic construct; identical to SEQ ID NO: 5
  source 1..33: mol_type = other DNA

SEQ ID NO: 19 - DNA, 35 bp, synthetic construct
  source 1..35: mol_type = other DNA
tttttagttgggctagctacaacgagttagtactc

SEQ ID NO: 20 - DNA, 33 bp, synthetic construct
  source 1..33: mol_type = other DNA
tcataattaggctagctacaacgatttagcata

SEQ ID NO: 21 - DNA, 33 bp, synthetic construct
  source 1..33: mol_type = other DNA
gaagtcgtgggctagctacaacgacagatacat

SEQ ID NO: 22 - DNA, 31 bp, synthetic construct
  source 1..31: mol_type = other DNA
gttcgttgtccgagccggacgacgaataatt

SEQ ID NO: 23 - DNA, 33 bp, synthetic construct; identical to SEQ ID NO: 9
  source 1..33: mol_type = other DNA

SEQ ID NO: 24 - DNA, 33 bp, synthetic construct
  source 1..33: mol_type = other DNA
atttatttaggctagctacaacgaaaaatttac

SEQ ID NO: 25 - DNA, 33 bp, synthetic construct
  source 1..33: mol_type = other DNA
tttttcgtgggctagctacaacgattctttttc

SEQ ID NO: 26 - DNA, 33 bp, synthetic construct; identical to SEQ ID NO: 20
  source 1..33: mol_type = other DNA

SEQ ID NO: 27 - DNA, 18 bp, Klebsiella pneumoniae
  source 1..18: mol_type = genomic DNA
aaatatctgacaacaggc

SEQ ID NO: 28 - DNA, 15 bp, synthetic construct
  source 1..15: mol_type = other DNA
ggctagctacaacga

SEQ ID NO: 29 - DNA, 14 bp, synthetic construct
  source 1..14: mol_type = other DNA
tccgagccggacga

SEQ ID NO: 30 - RNA, 17 nt, synthetic construct
  source 1..17: mol_type = other RNA
acccggtttgacttccg

SEQ ID NO: 31 - DNA, 33 bp, synthetic construct
  source 1..33: mol_type = other DNA
cagtttttgggctagctacaacgaaagctttcc

SEQ ID NO: 32 - DNA, 33 bp, synthetic construct
  source 1..33: mol_type = other DNA
catgagagaggctagctacaacgaaagacagca

SEQ ID NO: 33 - DNA, 31 bp, synthetic construct
  source 1..31: mol_type = other DNA
gaacgtggggctagctacaacgaatcgccga

SEQ ID NO: 34 - DNA, 32 bp, synthetic construct
  source 1..32: mol_type = other DNA
cggcgttaggctagctacaacgacactgtatt

SEQ ID NO: 35 - DNA, 33 bp, synthetic construct
  source 1..33: mol_type = other DNA
ttttgtaagggctagctacaacgatttccgtca

SEQ ID NO: 36 - DNA, 33 bp, synthetic construct; identical to SEQ ID NO: 2
  source 1..33: mol_type = other DNA

SEQ ID NO: 37 - DNA, 33 bp, synthetic construct
  source 1..33: mol_type = other DNA
cagtgtcagggctagctacaacgattttgtaag

SEQ ID NO: 38 - DNA, 31 bp, synthetic construct
  source 1..31: mol_type = other DNA
gtgtttcctccgagccggacgattagccaat

SEQ ID NO: 39 - DNA, 32 bp, synthetic construct
  source 1..32: mol_type = other DNA
ccgtcatgggctagctacaacgactgttgtca

SEQ ID NO: 40 - DNA, 33 bp, synthetic construct
  source 1..33: mol_type = other DNA
atagacttgggctagctacaacgattgtatgtg

SEQ ID NO: 41 - DNA, 32 bp, synthetic construct
  source 1..32: mol_type = other DNA
ccacggaaggctagctacaacgatgattggga

SEQ ID NO: 42 - DNA, 33 bp, synthetic construct
  source 1..33: mol_type = other DNA
tataaggtaggctagctacaacgattccggtaa

SEQ ID NO: 43 - DNA, 32 bp, synthetic construct
  source 1..32: mol_type = other DNA
tttggcgaggctagctacaacgatgcaagatt

SEQ ID NO: 44 - DNA, 33 bp, synthetic construct
  source 1..33: mol_type = other DNA
ccatttctaggctagctacaacgacatgcctac

SEQ ID NO: 45 - DNA, 33 bp, synthetic construct
  source 1..33: mol_type = other DNA
ggtggctaaggctagctacaacgaagggagata

SEQ ID NO: 46 - DNA, 33 bp, synthetic construct
  source 1..33: mol_type = other DNA
ttaaaggtgggctagctacaacgatcatcatgg

SEQ ID NO: 47 - DNA, 32 bp, synthetic construct
  source 1..32: mol_type = other DNA
gctcgtcgggctagctacaacgattggtatgg

SEQ ID NO: 48 - DNA, 33 bp, synthetic construct
  source 1..33: mol_type = other DNA
tctatttcgggctagctacaacgatcatccata

SEQ ID NO: 49 - DNA, 33 bp, synthetic construct
  source 1..33: mol_type = other DNA
acacggaaaggctagctacaacgagttgaatac

(19) United States
(12) Patent Application Publication
(10) Pub. No.: US 2026/0167960 A1
(43) Pub. Date: Jun. 18, 2026

(54) METHODS AND COMPOSITIONS INCLUDING CONJUGATED DNAZYMES

(71) Applicant: 1E Therapeutics Ltd., Rehovot (IL)

(72) Inventors: Alexander ROSENBERG, Rehovot (IL); Itzhak ZANDER, Rehovot (IL); Guy SHMUL, Rehovot (IL)

(73) Assignee: 1E Therapeutics Ltd., Rehovot (IL)

(21) Appl. No.: 19/127,109

(22) PCT Filed: Oct. 3, 2023
(86) PCT No.: PCT/IL2023/051055

§ 371 Date: May 4, 2025

Related U.S. Application Data
(60) Provisional application No. 63/425,406, filed on Nov. 15, 2022.

(51) Int. Cl. C12N 15/113; A61K 31/397; A61K 31/407; A61K 31/427; A61K 31/43; A61K 31/545; A61P 31/04

(52) U.S. Cl. CPC C12N 15/113; A61K 31/397; A61K 31/407; A61K 31/427; A61K 31/43; A61K 31/545; A61P 31/04; C12N 2310/127; C12N 2310/351

(57) ABSTRACT

Provided herein are DNAzymes conjugated to a penetration-enhancing moiety and methods of facilitating entry of DNAzymes into bacteria, utilizing same. Also provided are methods of targeting bacterial genes, methods of treating or inhibiting the progression of bacterial infections, and methods of increasing susceptibility of bacteria to an antibiotic, using the described conjugated DNAzymes, which are optionally capable of silencing at least one target gene of bacteria and/or rendering bacteria susceptible to antibiotic treatment.

### SEQUENCE LISTING STATEMENT

The instant application contains a Sequence Listing which has been submitted electronically in XML format and is hereby incorporated by reference in its entirety. Said XML copy, created on Sep. 14, 2023, is named P-621324-PC_SL.xml and is 32.9 Kilobytes in size.

### FIELD OF INTEREST

The present disclosure relates in general to DNAzymes conjugated to a penetration-enhancing moiety that contains a polycyclic ring system and an alkyl moiety; and methods of facilitating entry of DNAzymes into bacteria, utilizing the DNAzymes. Also described are methods of targeting bacterial genes, methods of treating or inhibiting the progression of bacterial infections, and methods of increasing susceptibility of bacteria to an antibiotic, using the described conjugated DNAzymes, which are optionally capable of silencing at least one target gene of bacteria and/or rendering bacteria susceptible to antibiotic treatment. The present disclosure also relates in general to methods of treating bacterial biofilm infections, utilizing DNAzymes conjugated to a penetration-enhancing moiety that contains a polycyclic ring system and an alkyl moiety, or conjugated to a polycyclic organic moiety having 3-5 rings.

### BACKGROUND

### DNAzymes

DNA enzymes (DNAzymes) are synthetic, catalytically-active DNA molecules that are able to specifically cleave target mRNA without requiring the involvement of cellular mechanisms such as the RNA-Induced Silencing Complex (RISC). DNAzymes have not been reported in nature and are typically generated by in-vitro selection.

DNAzymes typically consist of a catalytic core flanked by two arms that recognize its RNA target through Watson Crick base pairing and cleave RNA in a specific phosphodiester linkage. DNAzymes are a powerful tool for specific gene therapy due to their high specificity and catalytic efficiency.

The therapeutic potential of DNAzymes has been demonstrated in diverse settings, including in antibiotic-resistant bacterial infections. The studies published to date have generally utilized either plasmid transfection or physical disruption methods such as electroporation to enable penetration of the DNAzyme into the bacteria cell, which requires traversal of at least one cell membrane and a peptidoglycan-containing cell wall. Such methods may not be practicable in in-vivo therapeutic methods.

Improved methods of enhancing penetration of DNAzymes across bacterial cell walls are needed in the art.

### Biofilms

Bacteria can exist in a planktonic form, in which they exist as single cells, or in a sessile form, wherein they are organized into matrix-enclosed aggregates or microcolonies, also termed biofilms. Bacteria can transition between planktonic and biofilm lifestyles. Bacteria in biofilms are typically imbedded within a biopolymer matrix, which confers increased tolerance to multiple classes of antimicrobials and host defense mechanisms, relative to planktonic cells. Resistance to antibiotics and host defense is likely due to indolent growth and altered antimicrobial resistance mechanisms. Acute infections are considered more likely mediated by planktonic bacteria, whereas chronic infections resistant to antibiotic therapies often involve biofilms.

### SUMMARY

In certain aspects, provided herein are DNAzymes conjugated to a penetration-enhancing moiety and their use in treating a bacterial infection and potentiating antibiotic treatment in a subject in need thereof, and methods of facilitating entry of DNAzymes into bacteria.

In some aspects, provided herein is a DNAzyme conjugated to a penetration-enhancing moiety, wherein the penetration-enhancing moiety comprises a polycyclic ring system and an alkyl moiety. In some embodiments, the DNAzyme is conjugated to the polycyclic ring via a linker. In some aspects, the DNAzyme is directly conjugated to the polycyclic ring.

In some aspects, the described polycyclic ring system contains between 2-6 rings. In some aspects, 2-5 rings are present. In some aspects, 2-4 rings are present. In some aspects, 2-3 rings are present. In some aspects, 2 rings are present. In some aspects, 3 rings are present. In some aspects, 4 rings are present.

In some aspects, provided herein is use of a penetration-enhancing moiety in facilitating entry of a DNAzyme into a bacterium. In some aspects, provided herein is a DNAzyme conjugated to a penetration-enhancing moiety for use in treating a bacterial infection in a subject in need thereof. In some aspects, provided herein is a method of treating a bacterial infection in a subject in need thereof, comprising administering to the subject a DNAzyme conjugated to a penetration-enhancing moiety. In some aspects, provided herein is an article of manufacture comprising a DNAzyme conjugated to a penetration-enhancing moiety, being packaged in a packaging material and identified in print, in or on the packaging material, for use in treating a bacterial infection. In some aspects, the DNAzyme targets an RNA essential to viability of the bacteria. In some aspects, the DNAzyme reduces expression of the gene product of an RNA essential to viability of the bacteria. In some aspects, the DNAzyme targets an RNA product of an antibiotic resistance gene. In some aspects, the DNAzyme reduces expression of an antibiotic resistance protein.

In some aspects, provided herein is a DNAzyme conjugated to a penetration-enhancing moiety for use in treating a bacterial infection in a subject in need thereof. In some aspects, provided herein is a method of treating a bacterial infection in a subject in need thereof, comprising administering to the subject a DNAzyme conjugated to a penetration-enhancing moiety. In some aspects, provided herein is an article of manufacture comprising a DNAzyme conjugated to a penetration-enhancing moiety, being packaged in a packaging material and identified in print, in or on the packaging material, for use in treating a bacterial infection. In some aspects, the DNAzyme targets an RNA essential to viability of the bacteria. In some aspects, the DNAzyme reduces expression of the gene product of an RNA essential to viability of the bacteria. In some aspects, the DNAzyme targets an RNA product of an antibiotic resistance gene. In some aspects, the DNAzyme reduces expression of an antibiotic resistance protein.

In some aspects, provided herein is a DNAzyme conjugated to a penetration-enhancing moiety for use in inhibiting progression of a bacterial infection in a subject in need thereof. In some aspects, provided herein is a method of inhibiting progression of a bacterial infection in a subject in need thereof, comprising administering to the subject a DNAzyme conjugated to a penetration-enhancing moiety. In some aspects, provided herein is an article of manufacture comprising a DNAzyme conjugated to a penetration-enhancing moiety, being packaged in a packaging material and identified in print, in or on the packaging material, for use in inhibiting progression of a bacterial infection. In some aspects, the DNAzyme targets an RNA essential to viability of the bacteria. In some aspects, the DNAzyme reduces expression of the gene product of an RNA essential to viability of the bacteria. In some aspects, the DNAzyme targets an RNA product of an antibiotic resistance gene. In some aspects, the DNAzyme reduces expression of an antibiotic resistance protein.

In some aspects, provided herein is a DNAzyme conjugated to a penetration-enhancing moiety for use in increasing susceptibility of a bacterium to an antibiotic, in a subject having a bacterial infection. In some aspects, provided herein is a method of increasing susceptibility of a bacterium to an antibiotic in a subject in need thereof, comprising administering to the subject a DNAzyme conjugated to a penetration-enhancing moiety. In some aspects, provided herein is an article of manufacture comprising a DNAzyme conjugated to a penetration-enhancing moiety, being packaged in a packaging material and identified in print, in or on the packaging material, for use in increasing susceptibility of a bacterium to an antibiotic. In some aspects, the DNAzyme targets a messenger RNA of an antibiotic resistance gene of the bacteria. In some aspects, the DNAzyme reduces expression of the protein product of an antibiotic resistance gene.

In some aspects, provided herein is a DNAzyme conjugated to a penetration-enhancing moiety for use in potentiating an antibiotic, in a subject having a bacterial infection. In some aspects, provided herein is a method of potentiating an antibiotic in a subject in need thereof, comprising administering to the subject a DNAzyme conjugated to a penetration-enhancing moiety. In some aspects, provided herein is an article of manufacture comprising a DNAzyme conjugated to a penetration-enhancing moiety, being packaged in a packaging material and identified in print, in or on the packaging material, for use in potentiating an antibiotic. In some aspects, the DNAzyme targets a RNA that encodes an antibiotic resistance protein of the bacteria. In some aspects, the DNAzyme reduces expression of an antibiotic resistance protein.

In some aspects, provided herein is a DNAzyme conjugated to a penetration-enhancing moiety for use in reducing expression of a target bacterial RNA. In some aspects, provided herein is a method of reducing expression of a target bacterial RNA, comprising administering to the subject a DNAzyme conjugated to a penetration-enhancing moiety. In some aspects, provided herein is an article of manufacture comprising a DNAzyme conjugated to a penetration-enhancing moiety, being packaged in a packaging material and identified in print, in or on the packaging material, for use in reducing expression of a target bacterial RNA. In some aspects, the target RNA is essential to viability of the bacteria. In some aspects, the target RNA is an antibiotic resistance gene. In some embodiments, the target bacteria is disposed within a subject, who is, in some embodiments, a subject with a bacterial infection.

In some aspects, provided herein is a method of delivering a DNAzyme to the interior of a bacterial cell, comprising conjugating the DNAzyme to a penetration-enhancing moiety. In some aspects, the DNAzyme targets an RNA essential to viability of the bacteria. In some aspects, the DNAzyme reduces expression of the gene product of an RNA essential to viability of the bacteria. In some aspects, the DNAzyme targets an RNA product of an antibiotic resistance gene. In some aspects, the DNAzyme reduces expression of an antibiotic resistance protein.

In some aspects, provided herein is a method of delivering a DNAzyme across a bacterial cell wall, comprising conjugating the DNAzyme to a penetration-enhancing moiety. In some aspects, the DNAzyme targets an RNA essential to viability of the bacteria. In some aspects, the DNAzyme reduces expression of a protein essential to viability of the bacteria. In some aspects, the DNAzyme targets an RNA transcript of an antibiotic resistance gene. In some aspects, the DNAzyme reduces expression of an antibiotic resistance protein. In some embodiments, the cell wall comprises peptidoglycan.

In some aspects, provided herein is a DNAzyme conjugated to a penetration-enhancing moiety for use in reducing a biofilm presence on a medical device, in a subject in need thereof. In some aspects, provided herein is a method of reducing a biofilm presence on a medical device in a subject in need thereof, comprising administering to the subject a DNAzyme conjugated to a penetration-enhancing moiety. In some aspects, provided herein is an article of manufacture comprising a DNAzyme conjugated to a penetration-enhancing moiety, being packaged in a packaging material and identified in print, in or on the packaging material, for use in reducing a biofilm presence on a medical device. In some aspects, the DNAzyme targets an RNA essential to viability of the bacteria. In some aspects, the DNAzyme reduces expression of the gene product of an RNA essential to viability of the bacteria. In some aspects, the DNAzyme targets an RNA product of an antibiotic resistance gene. In some aspects, the DNAzyme reduces expression of an antibiotic resistance protein. Those skilled in the art will appreciate, in light of the present disclosure, that bacterial infections associated with biofilms are treatable with the described DNAzymes. In some aspects, the penetration-enhancing moiety comprises a polycyclic ring system and an alkyl moiety. In some aspects, the penetration-enhancing moiety is a polycyclic organic moiety. In some aspects, the penetration-enhancing moiety is a steroid.

In related aspects, the described DNAzymes target an RNA transcript. In further related aspects, the DNAzyme comprises, in 5′ to 3′ order: (i) a first substrate-binding domain (also referred to herein as the “5′ arm”) comprising a sequence that base pairs with a first region of the RNA transcript; (ii) a DNAzyme catalytic core; and (iii) a second substrate-binding domain (also referred to herein as the “3′ arm”) comprising a sequence that base pairs with a second region of the RNA transcript positioned 5′ to the first region of the RNA transcript. In yet further related embodiments, upon binding of the DNAzyme to the RNA transcript, the DNAzyme catalytic core cleaves the RNA transcript. In certain aspects, the DNAzyme is capable of silencing at least one target gene of a bacterium.

In certain aspects, there is provided a pharmaceutical composition comprising the described conjugated DNAzymes, and a pharmaceutically acceptable carrier or diluent.

In certain aspects, there is provided an article of manufacture comprising the described conjugated DNAzymes.

In certain aspects, there is provided an article of manufacture comprising the described conjugated DNAzymes, and an antibiotic.

In certain aspects, there is provided a method of treating or preventing a bacterial infection in a subject in need thereof, the method comprising administering to the subject the described DNAzymes, thereby treating or preventing the bacterial infection in the subject.

In certain aspects, there is provided a method of treating or preventing a bacterial infection in a subject in need thereof, the method comprising administering to the subject the described DNAzymes and an antibiotic, thereby treating or preventing the bacterial infection in the subject.

In certain aspects, there is provided a method of treating a bacterial biofilm associated with a medical device in a subject in need thereof, the method comprising administering to the subject the described DNAzymes and an antibiotic, thereby treating a bacterial biofilm associated with a medical device.

In certain aspects, the DNAzyme is a 10-23 type DNAzyme molecule.

In certain aspects, the DNAzyme is an 8-17 type DNAzyme molecule.

In certain aspects, the DNAzyme comprises a catalytic loop having the sequence: accegguuugacuuccg (SEQ ID NO: 30). In related aspects, the DNAzyme comprises modified nucleotides. In further related aspects, modified nucleotides comprise 2′-FANA.

In certain aspects, the bacterium is a Gram-positive bacterium.

In certain aspects, the bacterium is a Gram-negative bacterium.

In certain aspects, the bacterium is selected from the group consisting of Enterococcus faecium, Staphylococcus aureus, Klebsiella pneumoniae, Acinetobacter baumannii, Pseudomonas aeruginosa, Escherichia coli (E. coli) and an Enterobacter. In certain aspects, the bacterium is selected from E. coli and Klebsiella pneumoniae.

In certain embodiments, the described conjugated DNAzyme is administered to a subject in need together with an antibiotic. In certain aspects, the DNAzyme and the antibiotic are in a co-formulation. In other aspects, the DNAzyme and the antibiotic are in separate formulations.

In certain aspects, the subject is a human subject. In other aspects, the subject is a non-human subject.

In certain aspects, the antibiotic is a β-lactam. In other aspects, the antibiotic is a carbapenem. In other aspects, the antibiotic is a penicillin. In other aspects, the antibiotic is a cephalosporin. In other aspects, the antibiotic is a monobactam. In other aspects, the antibiotic is selected from the group consisting of penicillin, methicillin, oxacillin, cephalosporin, aztreonam, cefoxitin, carbapenem, imipenem and meropenem.

In certain aspects, the described DNAzyme comprises or consists of ribonucleotides, deoxyribonucleotides, or a combination thereof.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

The subject matter regarded as the conjugated deoxyribozymes (DNAzymes) and methods disclosed herein is particularly pointed out and distinctly claimed in the concluding portion of the specification. The conjugated DNAzymes and methods of use thereof, however, both as to organization and method of operation, together with objects, features, and advantages thereof, may best be understood by reference to the following detailed description when read with the accompanying drawings in which:

FIG. 1A illustrates a schematic illustration of the binding of 10-23 DNAzyme to its target RNA. FIG. 1B illustrates the conservation of the target region of the DNAzyme KPC-337 targeting the bla carbapenemase in clinical isolates of Klebsiella pneumoniae (ec number 3.5.2.6, uniport ID Q9F663).

FIG. 2A is a graph depicting the MIC₉₀ of meropenem in micrograms (mcg)/mL (vertical axis) for E. coli in the presence of increasing concentrations (horizontal axis) of conjugated DNAzymes. FIG. 2B is a graph depicting expression fold change of bla-KPC transcript levels (vertical axis) in the presence of increasing concentrations (horizontal axis) of conjugated DNAzymes. In FIGS. 2A-2B, DNAzymes conjugated to cholesterol or alpha-tocopherol are depicted in black and gray datasets, respectively. FIG. 2C is a graph depicting secreted beta-lactamase activity (horizontal axis) over time in minutes (vertical axis) in the presence of conjugated DNAzymes. “NT” depicts untreated bacteria. The double lines in each dataset represent biological duplicate samples.

FIG. 3A is a graph depicting the MIC₉₀ of meropenem in micrograms (mcg)/mL (vertical axis) for carbapenem-resistant K. pneumoniae (CRKP) in the presence of increasing concentrations (horizontal axis) of DNAzymes conjugated to cholesterol or alpha-tocopherol. FIG. 3B is a graph depicting expression fold change of bla-KPC transcript levels (vertical axis) in the presence of increasing concentrations (horizontal axis) of conjugated DNAzymes. Horizontal lines depict the mean of each dataset. In FIGS. 3A-B, black and gray datasets depict cholesterol-conjugated and tocopherol-conjugated DNAzymes, respectively. FIG. 3C is a graph depicting CFU/mL (vertical axis) following no treatment (ctrl) or treatment with scrambled (SCR; inactive) DNAzyme or DNAzymes conjugated to cholesterol or tocopherol (as indicated in the legend) for the time indicated in horizontal axis. Triangle and square symbols are superimposed.

FIG. 4 contains flow cytometry histograms depicting frequency (vertical axis) vs. mean fluorescent intensity (MFI) (horizontal axis) of carbapenem-resistant E. coli incubated with fluorescent DNAzymes that were unconjugated (left column) or conjugated to alpha-tocopherol (middle column) or vitamin B12 (right column) for 1, 2, or 3 hours (top, middle, and bottom rows, respectively).

FIG. 5A contains flow cytometry histograms of frequency (vertical axis) vs. mean fluorescent intensity (MFI) (horizontal axis) of CRKP incubated with fluorescent DNAzymes that were conjugated to alpha-tocopherol (left column) or cholesterol (middle column) or unconjugated (3′invdT; right column) for 3 or 5 hours (top and bottom rows, respectively). The internal vertical line in each plot demarcates negative-vs. positive-staining cells. FIG. 5B contains graphs of OD₅₉₅ (vertical axis) following crystal violet staining of K. pneumoniae or E. coli (top and bottom rows, respectively) after no treatment (NT); treatment with vehicle alone; or DNAzyme unconjugated (third and fifth rows) DNAzyme or conjugated with tocopherol (fourth and sixth rows) with the indicated concentrations of meropenem (rows from left to right in each series) of planktonic and biofilm bacteria (left and right columns, respectively). FIG. 5C is a photograph of the MBEC Assay® Kit utilized in the experiment of FIG. 5D. FIG. 5D contains graphs of OD₅₉₅ (vertical axis) of planktonic bacteria, or of biofilm after crystal violet staining (top and bottom rows, respectively) with no treatment (NT), treatment with vehicle alone, meropenem only, or DNAzyme conjugated with cholesterol or tocopherol (rows from left to right in each series). Left and right columns depict results of single or double treatment, respectively.

FIG. 6A is a plot of CFU/mL (vertical axis) of silicon-coated latex Foley catheter-associated bacterial biofilms administered the treatments indicated in the horizontal axis. FIG. 6B are charts depicting the CFU assay performed with or without 10 mcg/mL meropenem.

FIG. 7A is a picture of gram staining confirming colonization by CRKP of the lung tissue samples. Internalization of tocopherol-conjugated DNAzymes was shown by colocalization of fluorescent DNAzymes and bacterial colonies (FIG. 7B) and flow cytometry histograms depicting intracellular DNAzyme levels in bacteria isolated from the infected tissue (FIG. 7C).

### DETAILED DESCRIPTION

In the following detailed description, numerous specific details are set forth in order to provide a thorough understanding of the described conjugated DNAzymes. However, it will be understood by those skilled in the art that the conjugated DNAzymes and uses thereof may be practiced without these specific details. In other instances, well-known methods, procedures, and components have not been described in detail so as not to obscure the DNAzymes presented herein.

The present disclosure, in some embodiments thereof, relates to DNAzymes conjugated to a penetration-enhancing moiety, their use in treating a bacterial infection and potentiating antibiotic treatment in a subject in need thereof, and methods of facilitating entry of DNAzymes into bacteria.

In some embodiments, provided herein is a DNAzyme conjugated to a penetration-enhancing moiety, wherein the penetration-enhancing moiety comprises a polycyclic ring system and an alkyl moiety. In some embodiments, the DNAzyme is conjugated to the polycyclic ring via a linker. In some embodiments, the DNAzyme is directly conjugated to the polycyclic ring.

In some embodiments, provided herein is a DNAzyme conjugated to a described penetration-enhancing moiety, for use in treating a bacterial infection in a subject in need thereof. In some embodiments, provided herein is a method of treating a bacterial infection in a subject in need thereof, comprising administering to the subject a DNAzyme conjugated to a described penetration-enhancing moiety. In some embodiments, provided herein is an article of manufacture, comprising a DNAzyme conjugated to a described penetration-enhancing moiety, being packaged in a packaging material and identified in print, in or on the packaging material, for use in treating a bacterial infection. In some embodiments, the DNAzyme is capable of silencing a target gene of a bacterium. In some embodiments, the DNAzyme targets an RNA essential to viability of the bacteria. In some embodiments, the DNAzyme reduces expression of the gene product of an RNA essential to viability of the bacteria. In some embodiments, the DNAzyme targets an RNA product of an antibiotic resistance gene. In some embodiments, the DNAzyme reduces expression of an antibiotic resistance protein.

In some embodiments, provided herein is a DNAzyme conjugated to a described penetration-enhancing moiety, for use in inhibiting progression of a bacterial infection in a subject in need thereof. In some embodiments, provided herein is a method of inhibiting progression of a bacterial infection in a subject in need thereof, comprising administering to the subject a DNAzyme conjugated to a described penetration-enhancing moiety. In some embodiments, provided herein is an article of manufacture, comprising a DNAzyme conjugated to a described penetration-enhancing moiety, being packaged in a packaging material and identified in print, in or on the packaging material, for use in inhibiting progression of a bacterial infection. In some embodiments, the DNAzyme is capable of silencing a target gene of a bacterium. In some embodiments, the DNAzyme targets an RNA essential to viability of the bacteria. In some embodiments, the DNAzyme reduces expression of the gene product of an RNA essential to viability of the bacteria. In some embodiments, the DNAzyme targets an RNA product of an antibiotic resistance gene. In some embodiments, the DNAzyme reduces expression of an antibiotic resistance protein.

In some embodiments, the described penetration-enhancing moiety increases uptake of the DNAzyme into bacterial cells by at least 5-fold, relative to naked DNAzyme, during a 20-minute (min.) incubation at 37° C. In some embodiments, the moiety increases uptake of the DNAzyme into bacterial cells by at least 10-fold during a 20-min. incubation at 37° C. In some embodiments, the moiety increases uptake of the DNAzyme into bacterial cells by at least 20-fold during a 20-min. incubation at 37° C. In some embodiments, the moiety increases uptake of the DNAzyme into bacterial cells by at least 50-fold during a 20-min. incubation at 37° C. In some embodiments, the moiety increases uptake of the DNAzyme into bacterial cells by at least 100-fold during a 20-min. incubation at 37° C.

In some embodiments, provided herein is a DNAzyme conjugated to a described penetration-enhancing moiety, for use in increasing susceptibility of a bacterium to an antibiotic, in a subject having a bacterial infection. In some embodiments, provided herein is a method of increasing susceptibility of a bacterium to an antibiotic in a subject in need thereof, comprising administering to the subject a DNAzyme conjugated to a described penetration-enhancing moiety. In some embodiments, provided herein is an article of manufacture, comprising a DNAzyme conjugated to a described penetration-enhancing moiety, being packaged in a packaging material and identified in print, in or on the packaging material, for use in increasing susceptibility of a bacterium to an antibiotic. In some embodiments, the DNAzyme is capable of silencing a target gene of a bacterium. In some embodiments, the DNAzyme targets a messenger RNA of an antibiotic resistance gene of the bacteria. In some embodiments, the DNAzyme reduces expression of the protein product of an antibiotic resistance gene.

In some embodiments, the described conjugated DNAzyme decreases the minimal inhibitory concentration (MIC) of the utilized antibiotic by at least 30%, relative to the antibiotic administered without DNAzyme. In some embodiments, the conjugated DNAzyme decreases the MIC of the antibiotic by at least 50%, relative to the antibiotic administered alone. In some embodiments, the conjugated DNAzyme decreases the MIC of the antibiotic by at least 70%, relative to the antibiotic administered alone. In some embodiments, the conjugated DNAzyme decreases the MIC of the antibiotic by at least 80%, relative to the antibiotic administered alone. In some embodiments, the conjugated DNAzyme decreases the MIC of the antibiotic by at least 90%, relative to the antibiotic administered alone.

In some embodiments, the described conjugated DNAzyme decreases the minimal bactericidal concentration (MBC) of the utilized antibiotic by at least 30%, relative to the antibiotic administered without DNAzyme. In some embodiments, the conjugated DNAzyme decreases the MBC of the antibiotic by at least 50%, relative to the antibiotic administered alone. In some embodiments, the conjugated DNAzyme decreases the MBC of the antibiotic by at least 70%, relative to the antibiotic administered alone. In some embodiments, the conjugated DNAzyme decreases the MBC of the antibiotic by at least 80%, relative to the antibiotic administered alone. In some embodiments, the conjugated DNAzyme decreases the MBC of the antibiotic by at least 90%, relative to the antibiotic administered alone.

In some embodiments, provided herein is a DNAzyme conjugated to a described penetration-enhancing moiety, for use in potentiating an antibiotic, in a subject having a bacterial infection. In some embodiments, provided herein is a method of potentiating an antibiotic in a subject in need thereof, comprising administering to the subject a DNAzyme conjugated to a described penetration-enhancing moiety. In some embodiments, provided herein is an article of manufacture, comprising a DNAzyme conjugated to a described penetration-enhancing moiety, being packaged in a packaging material and identified in print, in or on the packaging material, for use in potentiating an antibiotic. In some embodiments, the DNAzyme is capable of silencing a target gene of a bacterium. In some embodiments, the DNAzyme targets an RNA that encodes an antibiotic resistance protein of the bacteria. In some embodiments, the DNAzyme reduces expression of an antibiotic resistance protein.

In some embodiments, provided herein is a DNAzyme conjugated to a described penetration-enhancing moiety, for use in reducing expression of a target bacterial RNA, in a subject having a bacterial infection. In some embodiments, provided herein is a method of reducing expression of a target bacterial RNA in a subject in need thereof, comprising administering to the subject a DNAzyme conjugated to a described penetration-enhancing moiety. In some embodiments, provided herein is an article of manufacture, comprising a DNAzyme conjugated to a described penetration-enhancing moiety, being packaged in a packaging material and identified in print, in or on the packaging material, for use in reducing expression of a target bacterial RNA. In some embodiments, the DNAzyme is capable of silencing a target gene of a bacterium. In some embodiments, the target RNA is essential to viability of the bacteria. In some embodiments, the target RNA is an antibiotic resistance gene.

In some embodiments, reducing expression comprises reducing a concentration of a target RNA by at least 30% compared to untreated bacteria. In some embodiments, reducing expression comprises reducing a concentration of a target RNA by at least 50% compared to untreated bacteria. In some embodiments, reducing expression comprises reducing a concentration of a target RNA by at least 70% compared to untreated bacteria. In some embodiments, reducing expression comprises reducing a concentration of a target RNA by at least 90% compared to untreated bacteria. In other embodiments, reducing expression comprises reducing a concentration of a protein product of a target RNA by at least 30% compared to untreated bacteria. In other embodiments, reducing expression comprises reducing a concentration of a protein product of a target RNA by at least 50% compared to untreated bacteria. In other embodiments, reducing expression comprises reducing a concentration of a protein product of a target RNA by at least 70% compared to untreated bacteria. In other embodiments, reducing expression comprises reducing a concentration of a protein product of a target RNA by at least 90% compared to untreated bacteria. In some embodiments, reducing expression denotes reducing a concentration of a target RNA. In some embodiments, the reduction is by at least 30%, at least 50%, at least 70%, at least 80%, at least 90%, or at least 95%, compared to untreated bacteria. In other embodiments, reducing expression denotes reducing a concentration of a protein product of a target RNA. In some embodiments, the reduction is by at least 30%, at least 50%, at least 70%, at least 80%, at least 90%, or at least 95%, compared to untreated bacteria.

In some embodiments, provided herein is a method of delivering a DNAzyme to the interior of a bacterial cell, comprising conjugating the DNAzyme to a penetration-enhancing moiety. In some embodiments, the DNAzyme targets an RNA essential to viability of the bacteria. In some embodiments, the DNAzyme reduces expression of the gene product of an RNA essential to viability of the bacteria. In some embodiments, the DNAzyme is capable of silencing a target gene of a bacterium. In some embodiments, the DNAzyme targets an RNA product of an antibiotic resistance gene. In some embodiments, the DNAzyme reduces expression of an antibiotic resistance protein.

In some embodiments, provided herein is a method of delivering a DNAzyme across a bacterial cell wall, comprising conjugating the DNAzyme to a penetration-enhancing moiety and contacting a bacterial cell with the conjugated DNAzyme. In some embodiments, the DNAzyme targets an RNA essential to viability of the bacteria. In some embodiments, the DNAzyme reduces expression of the gene product of an RNA essential to viability of the bacteria. In some embodiments, the DNAzyme targets an RNA product of an antibiotic resistance gene. In some embodiments, the DNAzyme reduces expression of an antibiotic resistance protein.

In some embodiments, provided herein are use of the described conjugated DNAzymes for targeting bacterial genes.

In some embodiments, the described polycyclic ring system contains between 2-6 rings. In some embodiments, 2-5 rings are present. In some embodiments, 2-4 rings are present. In some embodiments, 2-3 rings are present. In some embodiments, 2 rings are present. In some embodiments, 3 rings are present. In some embodiments, 4 rings are present.

Reference herein to direct conjugation refers to a covalent bond between 2 specified moieties without an intervening linking group.

As provided herein, the described moieties enable DNA enzymes (DNAzymes) to penetrate bacteria cell walls and subsequently target and cleave RNA transcripts of selected bacterial genes (Examples). In some embodiments, administering these DNAzymes to bacteria is cytotoxic to the bacteria. In some embodiments, administering these DNAzymes to bacteria renders the bacteria susceptible to antibiotics.

As provided herein, DNAzymes conjugated to tocopherols can successfully penetrate bacterial cell walls, potentiate antibiotic treatment, and reduce target bacterial RNA expression in several bacterial species. Moreover, they are able to penetrate established biofilms and achieve dispersal of the biofilm and growth inhibition of bacteria in biofilms. These findings have been observed in both in vitro conditions and ex vivo infection models. None of these effects are expected to be observed with unconjugated DNAzymes, which lack the ability to penetrate bacteria cell walls (Examples).

As mentioned, in some embodiments, the described penetration-enhancing moiety comprises a polycyclic ring system and an alkyl moiety. In some embodiments, with reference to Structure A, the penetration-enhancing moiety comprises a polycyclic ring system containing m rings, wherein m is at least 2; wherein each ring in the system contains n ring atoms, wherein n is independently between 5-8 for each ring; and wherein the polycyclic ring system is linked to an alkyl moiety. In some embodiments the polycyclic ring system is directly linked to the alkyl moiety.

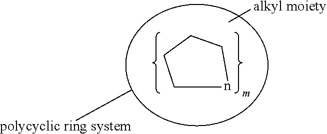

### Structure A; Penetration-Enhancing Moiety Comprising Polycyclic Ring System and Alkyl Moiety

### Polycyclic Ring Systems

In some embodiments, the described polycyclic ring system is an aromatic heterocyclic ring system. In some embodiments, the polycyclic ring system comprises at least one aromatic ring. In some embodiments, the presence of at least one aromatic ring confers aromatic character to the polycyclic ring system.

In some embodiments, a hydrogen atom in polycyclic ring system is replaced with another substituent. In some embodiments, 2-5 hydrogen atoms in polycyclic ring system are replaced with another substituent. In some embodiments, 2-4 hydrogen atoms in polycyclic ring system are replaced with another substituent. In some embodiments, 2-3 hydrogen atoms in polycyclic ring system are replaced with another substituent. In some embodiments, 2 hydrogen atoms in polycyclic ring system are replaced with another substituent. In some embodiments, the substituent(s) are hydroxyl moiety/ies, as in the depicted tocopherol compounds. In some embodiments, the substituent(s) are additional alkyl moiety/ies (in addition to the alkyl moiety described as attached to the polycyclic ring system). In some embodiments, the substituent(s) are methyl moiety/ies. In some embodiments, t multiple hydrogen atoms in polycyclic ring system are replaced with other substituents (which are, in some embodiments, any of the numbers of substituents mentioned hereinabove), and the substituent(s) are independently selected from hydroxyl moiety/ies and methyl moiety/ies.

In some embodiments, a hydrogen atom in polycyclic ring system is replaced with an additional alkyl moiety (in addition to the alkyl moiety described as attached to the polycyclic ring system). In some embodiments, the alkyl moiety and additional alkyl moiety are attached to the same atom of the polycyclic ring system. In some embodiments, the additional alkyl moiety is a methyl group, as in the depicted tocopherol compounds.

In some embodiments, the described heterocyclic ring system comprises a heterocyclic ring. In some embodiments, the heterocyclic ring system comprises at least one heterocyclic ring. In some embodiments, the heterocyclic ring comprises a least one heteroatom. In some embodiments, an oxygen atom is a ring substituent of the heterocyclic ring, as in the depicted tocopherol compounds. In some embodiments, no non-oxygen heteroatoms are present in the heterocyclic ring. In some embodiments, a single oxygen atom is the only heteroatom present in the heterocyclic ring.

In some embodiments, 2 rings are present in the polycyclic ring system that comprises a heterocyclic ring. In some embodiments, between 2-6 rings are present. In some embodiments, 2-5 rings are present. In some embodiments, 2-4 rings are present. In some embodiments, 2-3 rings are present. In some embodiments, 2 rings are present. In some embodiments, 3 rings are present. In some embodiments, 4 rings are present.

In some embodiments, the described polycyclic ring system comprises an aromatic ring and a non-aromatic ring. In some embodiments, the described polycyclic ring system comprises one or more aromatic rings and a non-aromatic ring. In some embodiments, the described polycyclic ring system comprises an aromatic ring and one or more non-aromatic rings. In some embodiments, the described polycyclic ring system comprises one or more aromatic rings and one or more non-aromatic rings. In some embodiments, 2 rings are present in a polycyclic ring system containing an aromatic ring(s) and a non-aromatic ring(s). In some embodiments, between 2-6 rings are present. In some embodiments, 2-5 rings are present. In some embodiments, 2-4 rings are present. In some embodiments, 2-3 rings are present. In some embodiments, 2 rings are present. In some embodiments, 3 rings are present. In some embodiments, 4 rings are present.

In some embodiments, wherein both an aromatic ring and a non-aromatic ring are present in the described polycyclic ring system, the described DNAzyme is conjugated to the aromatic ring via a linker. In some embodiments, wherein both an aromatic ring and a non-aromatic ring are present in the described polycyclic ring system, the described DNAzyme is directly conjugated to the aromatic ring. In some embodiments, if more than one aromatic ring is present in the polycyclic ring system, the DNAzyme is conjugated to one of the aromatic rings via a linker. In some embodiments, if more than one aromatic ring is present in the polycyclic ring system, the DNAzyme is directly conjugated to one of the aromatic rings.

In some embodiments, wherein both an aromatic ring and a non-aromatic ring are present in the described polycyclic ring system, the described non-aromatic ring is heterocyclic. In some embodiments, if more than one non-aromatic ring is present in the polycyclic ring system, at least one of the non-aromatic rings is heterocyclic. In some embodiments, the described non-aromatic ring comprises an oxygen atom as a ring substituent, as in the depicted tocopherol compounds. In some embodiments, at least one non-aromatic ring comprises an oxygen atom as a ring substituent. In some embodiments, every non-aromatic ring in the polycyclic ring system comprises an oxygen atom as a ring substituent. In some embodiments, the described heterocyclic ring comprises an oxygen atom as a ring substituent. In some embodiments, at least one heterocyclic ring comprises an oxygen atom as a ring substituent. In some embodiments, every heterocyclic ring in the polycyclic ring system comprises an oxygen atom as a ring substituent.

In some embodiments, wherein both an aromatic ring and a non-aromatic ring are present in the described polycyclic ring system, the described aromatic ring contains 6 ring atoms. In some embodiments, wherein at least one aromatic ring is present, at least one aromatic ring contains 6 ring atoms. In some embodiments, each aromatic ring in the polycyclic ring system contains 6 ring atoms.

In some embodiments, wherein both an aromatic ring and a non-aromatic ring are present in the described polycyclic ring system, the described non-aromatic ring contains 6 ring atoms. In some embodiments, wherein at least one non-aromatic ring is present, at least one non-aromatic ring contains 6 ring atoms. In some embodiments, each non-aromatic ring in the polycyclic ring system contains 6 ring atoms.

In some embodiments, wherein both an aromatic ring and a non-aromatic ring are present in the described polycyclic ring system, the described aromatic and non-aromatic rings each contain 6 ring atoms. In some embodiments, wherein at least one aromatic ring is present, at least one aromatic ring contains 6 ring atoms. In some embodiments, each aromatic ring in the polycyclic ring system contains 6 ring atoms. In some embodiments, wherein at least one non-aromatic ring is present, at least one non-aromatic ring contains 6 ring atoms. In some embodiments, each non-aromatic ring in the polycyclic ring system contains 6 ring atoms. In some embodiments, at least one aromatic ring and at least one non-aromatic ring contains 6 ring atoms. In some embodiments, each aromatic ring and each non-aromatic ring in the polycyclic ring system contains 6 ring atoms.

In some embodiments, wherein both an aromatic ring and a non-aromatic ring are present in the described polycyclic ring system, 2 rings are present in the polycyclic ring system. In some embodiments, between 2-6 rings are present. In some embodiments, 2-5 rings are present. In some embodiments, 2-4 rings are present. In some embodiments, 2-3 rings are present. In some embodiments, 2 rings are present. In some embodiments, 3 rings are present. In some embodiments, 4 rings are present.

In some embodiments, the described polycyclic ring system comprises a benzopyran system, for example as set forth in Structure XVII, where R¹ is as defined herein, and n=1-5). In some embodiments, the described polycyclic ring system is a benzopyran ring system. In some embodiments, the described polycyclic ring system consists of a benzopyran ring system.

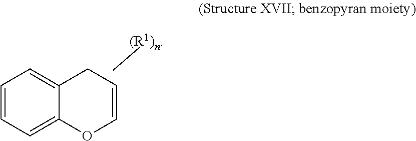

In some embodiments, the described polycyclic ring system comprises a benzodihydropyran ring system, for example as set forth in Structure XIX, where R¹ is as defined herein, and n=1-5). In some embodiments, the described polycyclic ring system is a benzodihydropyran ring system. In some embodiments, the described polycyclic ring system consists of a benzodihydropyran ring system.

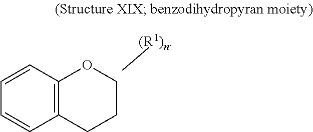

In some embodiments, the described polycyclic ring system comprises a chromane moiety (Structure XVIII). In some embodiments, the described polycyclic ring system is a chromane moiety. In some embodiments, the described polycyclic ring system consists of a chromane moiety. In some embodiments, the chromane moiety comprises a hydroxyl moiety. In some embodiments, the hydroxyl moiety is attached to the aromatic ring of the chromane moiety. In some embodiments, the hydroxyl moiety is attached to the 6-position of the chromane moiety (as depicted herein for the tocopherols). In some embodiments, the aromatic ring of the chromane moiety contains 1-3 methyl substituents. In some embodiments, the aforementioned embodiments may be freely combined.

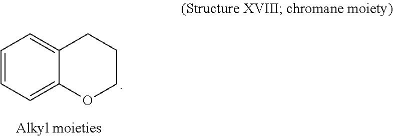

In some embodiments, the alkyl moiety conjugated to a polycyclic ring system is unsubstituted. In other embodiments, the alkyl moiety is substituted. In some embodiments, the alkyl moiety is a chain of 8-20 carbons, meaning that that total number of carbon atoms in the chain are 8-20. In some embodiments, the alkyl moiety is straight chained. In other embodiments, the alkyl moiety is branched. In some embodiments, the carbons of the alkyl moiety are replaced with up to 3 heteroatoms. In some embodiments, the heteroatoms are selected from nitrogen, oxygen, and sulfur; each of which represents a separate embodiment. In some embodiments, the chain is substituted with up to 3 functional groups at one or more positions, which may be any of the functional groups described herein, each of which represents a separate embodiment. In some embodiments, the described alkyl moiety is unbranched and unsubstituted. In some embodiments, the alkyl moiety is saturated, branched and unsubstituted, as in the depicted tocopherol compounds. In some embodiments, the saturated, branched and unsubstituted alkyl moiety has 12-20 total carbon atoms. In some embodiments, the saturated, branched and unsubstituted alkyl moiety has 13-19 total carbon atoms. In some embodiments, the saturated, branched and unsubstituted alkyl moiety has 14-18 total carbon atoms. In some embodiments, the saturated, branched and unsubstituted alkyl moiety has 15-17 total carbon atoms. In some embodiments, the saturated, branched and unsubstituted alkyl moiety has 16 total carbon atoms. In some embodiments, an additional alkyl moiety is attached to the polycyclic ring system. In some embodiments, the alkyl moiety and additional alkyl moiety are attached to the same atom of the polycyclic ring system. In some embodiments, the additional alkyl moiety is a methyl group, as in the depicted tocopherol compounds.

In some embodiments, the described penetration-enhancing moiety is a tocopherol. In some embodiments, the tocopherol is selected from the group consisting of tocopherol, alpha-tocopherol (Structure I), beta-tocopherol (Structure II), gamma-tocopherol (Structure III), and delta-tocopherol (Structure IV), and mixtures thereof.

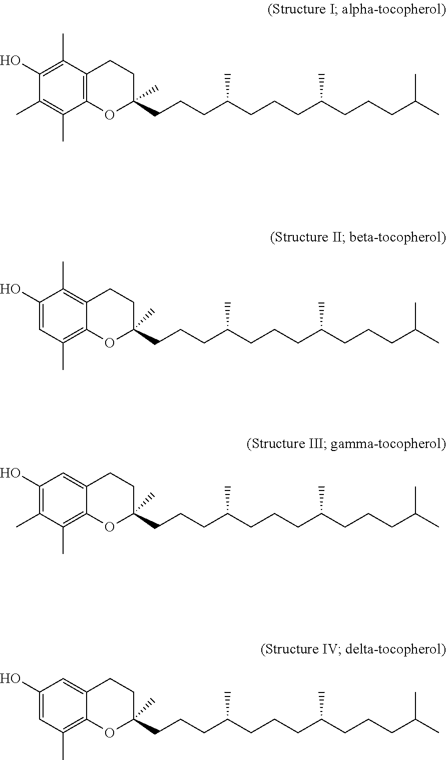 [SMILES: Cc1c(C)c2c(c(C)c1O)CC[C@@](C)(CCC[C@H](C)CCC[C@H](C)CCCC(C)C)O2 ; Cc1cc(O)c(C)c2c1O[C@](C)(CCC[C@H](C)CCC[C@H](C)CCCC(C)C)CC2 ; Cc1c(O)cc2c(c1C)O[C@](C)(CCC[C@H](C)CCC[C@H](C)CCCC(C)C)CC2 ; Cc1cc(O)cc2c1O[C@](C)(CCC[C@H](C)CCC[C@H](C)CCCC(C)C)CC2]

In some embodiments, the penetration-enhancing moiety can be attached to any nucleotide in the DNAzyme molecule. In some embodiments, the penetration-enhancing moiety is attached to the 3′ terminal nucleotide of the DNAzyme. In some embodiments, the penetration-enhancing moiety is attached to the 5′ terminal nucleotide of the DNAzyme. In some embodiments, an internal conjugate may be attached directly or indirectly through a linker to a nucleotide at a 2′ position of the ribose group, or to another suitable position.

In some embodiments, the penetration-enhancing moiety can be attached to any nucleotide within the DNAzyme molecule, as long as the silencing activity (catalytic activity) of the DNAzyme is not compromised.

In some embodiments, disclosed herein is a DNAzyme conjugated to a penetration-enhancing moiety, wherein said penetration-enhancing moiety comprises a benzodihydropyran ring system and an alkyl moiety, and wherein said DNAzyme is conjugated to said benzodihydropyran ring system. In some embodiments, said benzodihydropyran ring system comprises a chromane moiety. In some embodiments of a DNAzyme, an alkyl moiety comprises a chain of 8-20 carbons. In certain embodiments of a DNAzyme, a penetration-enhancing moiety comprises a tocopherol. In certain embodiments of a DNAzyme, a penetration-enhancing moiety comprises alpha-tocopherol. In some embodiments, the conjugated DNAzymes further comprises a linker conjugating the DNAzyme to the benzodihydropyran ring system. In some embodiments of a conjugated DNAzyme, a benzodihydropyran ring system or a linker is attached to the 3′ terminal nucleotide of said DNAzyme. In some embodiments of a conjugated DNAzyme, a benzodihydropyran ring system or a linker is attached to the 5′ terminal nucleotide of said DNAzyme. In some embodiments of a conjugated DNAzyme, a benzodihydropyran ring system or a linker is attached to the 3′ terminal nucleotide of said DNAzyme. In some embodiments of a conjugated DNAzyme, a benzodihydropyran ring system or a linker is attached to the 5′ or 3′ terminal nucleotide of said DNAzyme. In some embodiments of a conjugated DNAzyme comprising a linker, the linker comprises Triethylene glycol (TEG). In some embodiments of a conjugated DNAzyme, the DNAzyme is a 10-23-type DNAzyme molecule.

### Targeting and Treatment of Bacterial Biofilms

In some embodiments, provided herein is a DNAzyme conjugated to a penetration-enhancing moiety for use in treating a bacterial biofilm associated with a medical device, in a subject in need thereof. In some embodiments, provided herein is a method of treating a bacterial biofilm associated with a medical device in a subject in need thereof, comprising administering to the subject a DNAzyme conjugated to a penetration-enhancing moiety. In some embodiments, provided herein is an article of manufacture comprising a DNAzyme conjugated to a penetration-enhancing moiety, being packaged in a packaging material and identified in print, in or on the packaging material, for use in treating a bacterial biofilm associated with a medical device. In some embodiments, the DNAzyme targets an RNA essential to viability of the bacteria. In some embodiments, the DNAzyme reduces expression of the gene product of an RNA essential to viability of the bacteria. In some embodiments, the DNAzyme targets an RNA product of an antibiotic resistance gene. In some embodiments, the DNAzyme reduces expression of an antibiotic resistance protein. In some embodiments, the biofilm comprises an exopolysaccharide matrix. Those skilled in the art will appreciate, in light of the present disclosure, that bacterial infections associated with biofilms are treatable with the described DNAzymes. In some embodiments, the penetration-enhancing moiety comprises a polycyclic ring system and an alkyl moiety. Non-limiting examples of moieties comprising a polycyclic ring system and an alkyl moiety are described herein. In some embodiments, the penetration-enhancing moiety is a polycyclic organic moiety having 3-5 rings. In some embodiments, the penetration-enhancing moiety is a steroid. Non-limiting examples of polycyclic organic moieties having multiple rings, and steroids, are described herein.

There is also provided herein a method of reducing a biofilm presence on a medical device in a subject in need thereof, comprising treating the subject with a DNAzyme conjugated to a penetration-enhancing moiety that comprises a polycyclic ring system and an alkyl moiety, thereby reducing a biofilm presence on a medical device. In some embodiments, the polycyclic ring system has 2 rings. In some embodiments, the polycyclic ring system consists of 2 rings. There is also provided herein a method of reducing a biofilm presence on a medical device in a subject in need thereof, comprising treating the subject with a DNAzyme conjugated to a polycyclic organic moiety having 3-5 rings, thereby reducing a biofilm presence on a medical device. In some embodiments, the biofilm-associated bacteria are in an abscess. In other embodiments, the biofilm-associated bacteria form a coating on a medical device, for example a catheter. In some embodiments, the quantity of biofilm-associated bacteria on the medical device is reduced. In some embodiments, the quantity is reduced to a non-pathological level. In some embodiments, the medical device is disposed in the subject.

There is also provided herein a method of dispersing a biofilm on a medical device in a subject in need thereof, comprising treating the subject with a DNAzyme conjugated to a penetration-enhancing moiety that comprises a polycyclic ring system and an alkyl moiety, thereby dispersing a biofilm on a medical device. In some embodiments, the polycyclic ring system has 2 rings. In some embodiments, the polycyclic ring system consists of 2 rings. There is also provided herein a method of dispersing a biofilm on a medical device in a subject in need thereof, comprising treating the subject with a DNAzyme conjugated to a polycyclic organic moiety having 3-5 rings, thereby dispersing a biofilm on a medical device. In some embodiments, the biofilm-associated bacteria are in an abscess. In other embodiments, the biofilm-associated bacteria form a coating on a medical device, for example a catheter. In some embodiments, the quantity of biofilm-associated bacteria on the medical device is reduced. In some embodiments, the quantity is reduced to a non-pathological level. In some embodiments, the medical device is disposed in the subject.

There is also provided herein a method of treating an infection caused by biofilm-associated bacteria in a subject in need thereof, comprising treating the subject with a DNAzyme conjugated to a penetration-enhancing moiety that comprises a polycyclic ring system and an alkyl moiety, thereby treating an infection caused by biofilm-associated bacteria. In some embodiments, the polycyclic ring system has 2 rings. In some embodiments, the polycyclic ring system consists of 2 rings. There is also provided herein a method of treating an infection caused by biofilm-associated bacteria in a subject in need thereof, comprising treating the subject with a DNAzyme conjugated to a polycyclic organic moiety having 3-5 rings, thereby treating an infection caused by biofilm-associated bacteria. In some embodiments, the biofilm-associated bacteria are in an abscess. In other embodiments, the biofilm-associated bacteria form a coating on a medical device, for example a catheter. In some embodiments, the medical device is disposed in the subject.

There is also provided herein a method of treating an infection in a subject with a medical device, wherein the infection is caused by a bacterial biofilm associated with the medical device, comprising treating the subject with a DNAzyme conjugated to a penetration-enhancing moiety that comprises a polycyclic ring system and an alkyl moiety, thereby treating an infection in a subject with a medical device. In some embodiments, the polycyclic ring system has 2 rings. In some embodiments, the polycyclic ring system consists of 2 rings. There is also provided herein a method of treating an infection in a subject with a medical device, wherein the infection is caused by a bacterial biofilm associated with the medical device, comprising treating the subject with a DNAzyme conjugated to a polycyclic organic moiety having 3-5 rings, thereby treating an infection in a subject with a medical device. In some embodiments, the biofilm-associated bacteria are in an abscess. In other embodiments, the biofilm-associated bacteria form a coating on a medical device, for example a catheter. In some embodiments, the medical device is disposed in the subject.

There is also provided herein a method of treating a bacterial infection in a subject in need thereof, wherein the bacteria are in a biofilm, comprising treating the subject with a DNAzyme conjugated to a penetration-enhancing moiety that comprises a polycyclic ring system and an alkyl moiety. In some embodiments, the polycyclic ring system has 2 rings. In some embodiments, the polycyclic ring system consists of 2 rings. There is also provided herein a method of treating a bacterial infection in a subject in need thereof, wherein the bacteria are in a biofilm, comprising treating the subject with a DNAzyme conjugated to a polycyclic organic moiety having 3-5 rings. In some embodiments, the biofilm-associated bacteria are in an abscess. In other embodiments, the biofilm-associated bacteria form a coating on a medical device, for example a catheter. In some embodiments, the medical device is disposed in the subject.

As provided herein, conjugated DNAzymes are able to successfully reverse biofilm formation on silicon-coated latex Foley catheters, which is a model system intended to stimulate in-vivo catheter-associated biofilm infections. This achievement was observed with a single 2-hour perfusion of conjugated DNAzymes+a subclinical dose of antibiotic (Example 5). Without wishing to be bound by theory, it is possible that short-term exposure of DNAzyme and antibiotic may be further potentiated by a repeat exposure to antibiotic (which is also accomplished by antibiotic perfusion). The findings presented herein may be consistent with weakening of the cell wall following treatment with an antibiotic targeting bacterial cell wall synthesize and DNAzyme targeting a resistance gene to said antibiotic. The bacteria then exhibit increased sensitivity to subsequent antibiotic treatment (with the same or a different antibiotic) administered at least 1-2 hours after the initial treatment of DNAzyme+antibiotic.

In some embodiments, the medical device is an implantable device. In some embodiments, the implantable device is a medical catheter. In some embodiments, the implantable device is selected from the group consisting of an orthopedic device, a surgical mesh, an artificial kidney, a cardiovascular device, a corneal implant, an ear ventilation tube, an aneurysm coil, a substitute skin graft, an intraocular lens, a vital sign monitor, a drug depot device, a neurostimulator, a silicone implant (e.g., a soft tissue silicone implant), a dental implant, a dental prosthetic, and a hernia mesh. In some embodiments, the medical device is selected from the group consisting of an embolic protection device, a ureter renal biliary stent, a urethral sling, a gastric bypass balloon, a gastric pacemaker, an insulin pump, a penile implant, an intrauterine contraceptive device, a cochlear implant, and a voice restoration device.

In some embodiments, the catheter is selected from urinary catheter, a central vascular catheter, an intravascular catheter (e.g., an arterial catheter, a central venous or PICC catheter, or a peripheral venous catheter), a peritoneal dialysis catheter, a shunt, an intubation tube (e.g., an endotracheal tube), and a gastric feeding tube (e.g., a nasogastric feeding tube).

In some embodiments, the orthopedic device is selected from bone cement; a hip implant; an orthopedic rod, plate, screw, or pin; a synthetic bone graft, a knee implant, a shoulder implant, another joint replacement or prosthesis, and a spinal disc implant.

In some embodiments, the cardiovascular device is selected from a cardiac valve, a cardiac stent, an implantable defibrillator, a heart ventricular assist device, and a pacemaker.

In some embodiments, the described bacterial biofilm comprises an infection of a bacterium selected from Enterococcus faecalis, Staphylococcus aureus, Staphylococcus epidermidis, Streptococcus viridans, E. coli, Klebsiella pneumoniae, Proteus mirabilis, and Pseudomonas aeruginosa. In some embodiments, the bacterium is selected from S. aureus and S. epidermidis.

### Polycyclic Organic Moieties Having 3-5 Rings

As described herein, a DNAzyme conjugated to a polycyclic organic moiety having 3-5 rings can be used to treat infections with a bacterial biofilm.

In some embodiments, the described polycyclic organic moiety comprises 3-6 rings. In some embodiments, 3-5 rings are present. In some embodiments, 3-4 rings are present. In some embodiments, 4-5 rings are present. In some embodiments, 4 rings are present.

In some embodiments, the polycyclic organic moiety is directly conjugated to the DNAzyme. In other embodiments, the polycyclic organic moiety is conjugated to the DNAzyme via a linker.

In some embodiments, the described polycyclic organic moiety is a steroid, used herein to refer to compounds that contain an optionally substituted gonane structure (V). In some embodiments, the ring systems of the gonane structure are modified with up to 3 double bonds. In some embodiments, the ring systems of the gonane structure are modified with up to 3 heteroatoms. In some embodiments, the heteroatoms are selected from nitrogen, oxygen, and sulfur; each of which represents a separate embodiment. In some embodiments, the gonane structure is substituted with up to 3 additional functional groups at one or more of positions 1-17. In some embodiments, the functional groups are selected from OH, NO₂, NH₃, F, Cl, Br, I, NR₃, C(O)NR₃, unsubstituted alkyl, substituted alkyl, unsubstituted alkenyl, substituted alkenyl, unsubstituted alkynyl, substituted alkynyl, unsubstituted alkoxy, or substituted alkoxy; wherein R is, independently in each instance, H, unsubstituted alkyl, or substituted alkyl; each of which represents a separate embodiment. In some embodiments, substituted alkyl, substituted alkenyl, or substituted alkynyl contains 1-5 substitutions independently selected from OH, NO₂, NH₃, F, Cl, Br, I, NR₃, C(O)NR₃, where R is defined as above; each of which represents a separate embodiment. The above modifications may be freely combined, and each combination represents a separate embodiment.

In some embodiments, the steroid is directly conjugated to the DNAzyme. In other embodiments, the steroid is conjugated to the DNAzyme via a linker.

In some embodiments, the junction of the A and B rings has a trans configuration. In other embodiments, the junction of the A and B rings has a cis configuration.

In some embodiments, the steroid is physiologically acceptable.

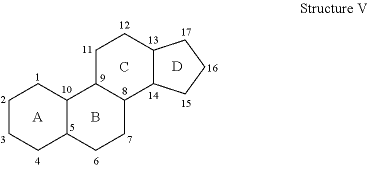

In some embodiments, the described polycyclic organic moiety has the following structure VI:

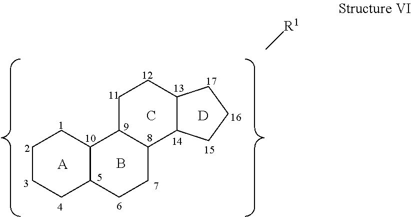

where n=1-3; and
R¹ is, independently in each instance, OH, NO₂, NH₃, F, Cl, Br, I, N(R²)₃, C(O)N N(R²)₃, unsubstituted alkyl, substituted alkyl, unsubstituted alkenyl, substituted alkenyl, unsubstituted alkynyl, substituted alkynyl, unsubstituted alkoxy, substituted alkoxy; wherein R² is, independently in each instance, H, unsubstituted alkyl, or substituted alkyl; each of which represents a separate embodiment. In some embodiments, substituted alkyl, substituted alkenyl, or substituted alkynyl contains 1-5 substitutions independently selected from OH, NO₂, NH₃, F, Cl, Br, I, N N(R²)₃, C(O)N N(R²)₃, where R² is defined as above; each of which represents a separate embodiment.

As used herein, “alkyl” refers to a monovalent saturated hydrocarbon moiety. In some embodiments, an alkyl moiety is linear. In some embodiments, an alkyl moiety is branched. In some embodiments, an alkyl moiety has between 1-20 carbon atoms. In some embodiments, an alkyl moiety has between 1-12 carbon atoms. In some embodiments, an alkyl moiety has between 1-8 carbon atoms. In some embodiments, an alkyl moiety has between 1-6 carbon atoms. In some embodiments, an alkyl moiety has between 1-4 carbon atoms. In some embodiments, an alkyl moiety has between 1-3 carbon atoms. Examples of alkyl moieties include, but are not limited to methyl, ethyl, npropyl, i-propyl, n-butyl, s-butyl, t-butyl, i-butyl, a pentyl moiety, a hexyl moiety, cyclopropyl, cyclobutyl, cyclopentyl, and cyclohexyl. Each of the aforementioned possibilities represents a separate embodiment.

As used herein, “alkenyl” refers to a monovalent hydrocarbon moiety containing at least two carbon atoms and one or more nonaromatic carbon-carbon double bonds. In some embodiments, an alkenyl moiety is linear. In some embodiments, an alkenyl moiety is branched. In some embodiments, an alkenyl moiety has between 1-12 carbon atoms. In some embodiments, an alkenyl moiety has between 1-8 carbon atoms. In some embodiments, an alkenyl moiety has between 1-6 carbon atoms. In some embodiments, an alkenyl moiety has between 1-4 carbon atoms. In some embodiments, an alkenyl moiety has between 1-3 carbon atoms. Examples of alkenyl moieties include, but are not limited to vinyl, propenyl, butenyl, and cyclohexenyl. Each of the aforementioned possibilities represents a separate embodiment.

As used herein, “alkynyl” refers to a monovalent hydrocarbon radical moiety containing at least two carbon atoms and one or more carbon-carbon triple bonds. In some embodiments, an alkynyl moiety is branched. In some embodiments, an alkynyl moiety has between 1-20 carbon atoms. In some embodiments, an alkynyl moiety has between 1-12 carbon atoms. In some embodiments, an alkynyl moiety has between 1-8 carbon atoms. In some embodiments, an alkynyl moiety has between 1-6 carbon atoms. In some embodiments, an alkynyl moiety has between 1-4 carbon atoms. In some embodiments, an alkynyl moiety has between 1-3 carbon atoms. Examples of alkynyl moieties include, but are not limited to ethynyl, propynyl, and butynyl. Each of the aforementioned possibilities represents a separate embodiment.

As used herein, “alkoxy” refers to a monovalent and saturated hydrocarbon moiety, wherein the hydrocarbon includes a single bond to an oxygen atom, e.g., CH₃CH₂—O. for ethoxy. Alkoxy substituents bond to the compound which they substitute through this oxygen atom of the alkoxy substituent. In some embodiments, an alkoxy moiety is linear. In some embodiments, an alkoxy moiety is branched. In some embodiments, an alkoxy moiety has between 1-20 carbon atoms. In some embodiments, an alkoxy moiety has between 1-12 carbon atoms. In some embodiments, an alkoxy moiety has between 1-8 carbon atoms. In some embodiments, an alkoxy moiety has between 1-6 carbon atoms. In some embodiments, an alkoxy moiety has between 1-4 carbon atoms. In some embodiments, an alkyl moiety has between 1-3 carbon atoms. Examples of alkoxy moieties include, but are not limited to methoxy, ethoxy, n-propoxy, i-propoxy, n-butoxy, s-butoxy, t-butoxy, i-butoxy, a pentoxy moiety, a hexoxy moiety, cyclopropoxy, cyclobutoxy, cyclopentoxy, and cyclohexoxy. Each of the aforementioned possibilities represents a separate embodiment.

In some embodiments, the steroid is a gonane alcohol. In some embodiments, the gonane alcohol is physiologically acceptable. In some embodiments, a sterol is a molecule derived from gonane by replacement of a hydrogen atom at any of positions 1-17 by a hydroxyl group.

In some embodiments, the steroid is a sterol (Structure VII). In some embodiments, the steroid differs from gonane by replacement of a hydrogen atom in position 3 by a hydroxyl group. In some embodiments, the steroid differs from gonane by replacement of a hydrogen atom in position 10 by a methyl group. In some embodiments, the steroid differs from gonane by replacement of a hydrogen atom in position 13 by a methyl group. In some embodiments, the steroid differs from gonane by replacement of a hydrogen atom in position 17 by an alkyl group. In some embodiments, the steroid differs from gonane by (a) replacement of a hydrogen atom in position 3 by a hydroxyl group; (b) replacement of a hydrogen atom in position 10 by a methyl group; (c) replacement of a hydrogen atom in position 13 by a methyl group; and (d) replacement of a hydrogen atom in position 17 by an alkyl group, each of which, alone or in combination, represents a separate embodiment. In some embodiments, the steroid has at least 2 of aforementioned elements (a)-(d). In some embodiments, the steroid has at least 3 of aforementioned elements (a)-(d). In some embodiments, the steroid has all of aforementioned elements (a)-(d). The above modifications may be freely combined, and each combination represents a separate embodiment.

In some embodiments, the junction of the A and B rings has a trans configuration. In other embodiments, the junction of the A and B rings has a cis configuration.

In some embodiments, the sterol is directly conjugated to the DNAzyme. In other embodiments, the sterol is conjugated to the DNAzyme via a linker.

In some embodiments, the sterol is physiologically acceptable

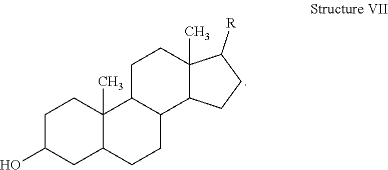

In some embodiments, a sterol is defined as per CHEBI:15889.

In some embodiments, the described polycyclic organic moiety has Structure VIII:

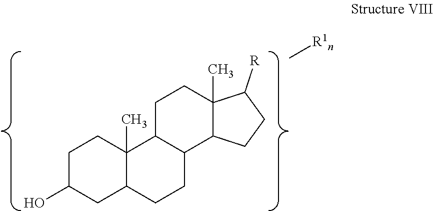

where n=1-3; and
where R¹ is defined as set forth hereinabove.

In some embodiments, the steroid is cholesterol (Structure IX; PubChem CID 5997), cholestan-3-ol (Structure X; PubChem CID 10992748), or a derivative of cholesterol or cholestan-3-ol, each of which represents a separate embodiment. In some embodiments, the sterol comprises a 3beta-hydroxy group. In some embodiments, the sterol has a double bond at the 5,6-position. In some embodiments, the sterol comprises an alkyl group at position 17. In some embodiments, the sterol comprises a 3beta-hydroxy group; a double bond at the 5,6-position; and an alkyl group at position 17. In some embodiments, the alkyl group contains 5-8 carbon atoms. Cholesterol is a non-limiting example of a cholestane having a 3beta-hydroxy group; a double bond at the 5,6-position; and an alkyl group at position 17.

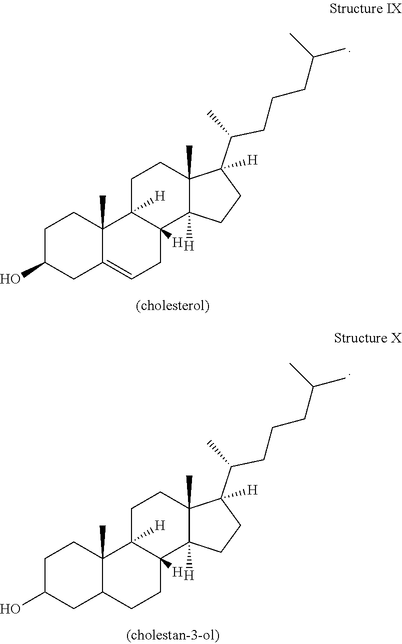

In some embodiments, the cholesterol, cholestan-3-ol, or derivative thereof is directly conjugated to the DNAzyme. In other embodiments, the cholesterol, cholestan-3-ol, or derivative thereof is conjugated to the DNAzyme via a linker.

In some embodiments, the ring systems of the derivative of cholesterol or cholestan-3-ol are modified with up to 3 double bonds (3 additional double bonds, in the case of a cholesterol derivative). In some embodiments, the ring systems of the gonane structure are modified with up to 3 heteroatoms. In some embodiments, the heteroatoms are selected from nitrogen, oxygen, and sulfur; each of which represents a separate embodiment. In some embodiments, as depicted in Structures XI and XII, the cholesterol or cholestan-3-ol structure is substituted with up to 3 additional functional groups at one or more positions, which may be any of the functional groups described herein, each of which represents a separate embodiment. In Structures XI and XII, n and R¹ are defined as set forth hereinabove.

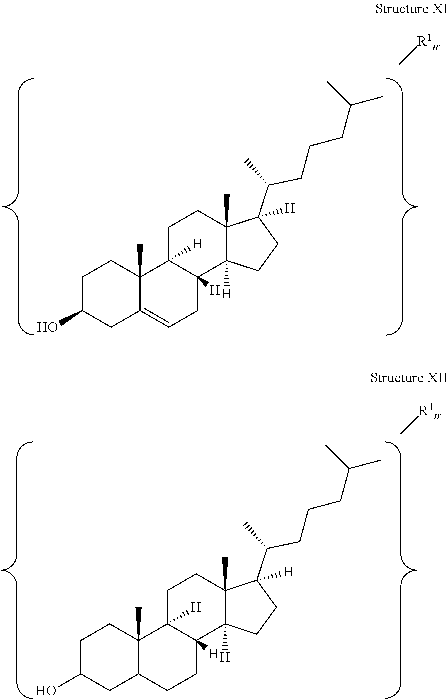

In some embodiments, the steroid is cholestane (Structure XIII; PubChem CID 637620) or a derivative of cholestane. In some embodiments, the ring systems of the derivative of cholestane are modified with up to 3 double bonds. In some embodiments, the ring systems of the gonane structure are modified with up to 3 heteroatoms. In some embodiments, the heteroatoms are selected from nitrogen, oxygen, and sulfur; each of which represents a separate embodiment. In some embodiments, the cholestane structure is substituted with up to 3 additional functional groups at one or more of positions 1-17, which may be any of the functional groups described herein, each of which represents a separate embodiment.

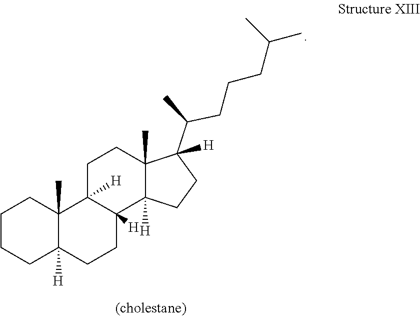

In some embodiments, the cholestane or derivative thereof is directly conjugated to the DNAzyme. In other embodiments, the cholestane or derivative thereof is conjugated to the DNAzyme via a linker.

In some embodiments, the ring systems of the derivative of cholestane are modified with up to 3 double bonds. In some embodiments, the ring systems of the gonane structure are modified with up to 3 heteroatoms. In some embodiments, the heteroatoms are selected from nitrogen, oxygen, and sulfur; each of which represents a separate embodiment. In some embodiments, as depicted in Structure X, the cholestane structure is substituted with up to 3 additional functional groups at one or more positions, which may be any of the functional groups described herein, each of which represents a separate embodiment. In the below Structure XIV, n and R¹ are defined as set forth hereinabove.

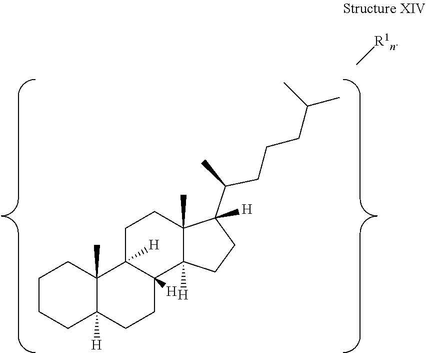

In some embodiments, the described sterol is selected from the group consisting of cholesterol, β-sitosterol, campesterol, stigmasterol, and ergosterol; and derivatives of any of aforementioned compounds with 2 or fewer substitutions.

In some embodiments, the polycyclic organic moiety is a bile acid or bile alcohol. In some embodiments, the terms bile acids and bile alcohols refer to steroids with a core structure of seventeen carbon atoms arranged in four fused rings, i.e., three cyclohexane rings (rings A-C) and one cyclopentane ring (ring D), together with a five or eight carbon sidechain terminating in a carboxylic acid group (or hydroxyl in the bile alcohols). In related embodiments, bile acids and bile alcohols further contain hydroxyl groups at positions C3, C7 and C12, and methyl groups at in positions C18 and C19. In related embodiments, the junction of the A and B rings of bile acids has a cis or chair configuration. In certain embodiments, the bile acid or bile alcohol is selected from a C₂₇ bile alcohols, a C₂₇ bile acid, and a C₂₄ bile acid.

In some embodiments, the bile alcohol is a C₂₇ bile alcohol. In some embodiments, the C₂₇ bile alcohol is physiologically acceptable.

In some embodiments, the bile acid is a C₂₇ bile acid. In some embodiments, the C₂₇ bile acid is physiologically acceptable.

In some embodiments, the bile acid is a C₂₄ bile acid. In some embodiments, the C₂₄ bile acid is physiologically acceptable.

In some embodiments, the described sterol is a phytosterol, non-limiting examples of which include β-sitosterol, campesterol, stigmasterol, stigmastanol, campestanol, and brassicasterol. In some embodiments, the phytosterol is physiologically acceptable.

In some embodiments, the described steroid is a physiologically acceptable steroid selected from the group consisting of androstane, cholestane, gorgostane, bufanolide, ergostane, poriferastane, campestane, estrane, pregnane, cardanolide, furostan, spirostan, cholane, gonane, and stigmastane.

In some embodiments, the penetration-enhancing moiety (which, as described herein, may comprise a polycyclic ring system and an alkyl moiety) can be attached to any nucleotide in the DNAzyme molecule. In some embodiments, the penetration-enhancing moiety is attached to the 3′ terminal nucleotide of the DNAzyme. In some embodiments, the penetration-enhancing moiety is attached to the 5′ terminal nucleotide of the DNAzyme. In some embodiments, the penetration-enhancing moiety is attached to an internal nucleotide. In some embodiments, the penetration-enhancing moiety is attached at a 2′ position of a ribose group of a nucleotide. In some embodiments, the penetration-enhancing moiety is attached to another position of a nucleotide. In some embodiments, the penetration-enhancing moiety can be attached to any nucleotide within the DNAzyme molecule, as long as the silencing activity (catalytic activity) of the DNAzyme is not compromised.

In some embodiments, the polycyclic organic moiety having 3-5 rings can be attached to any nucleotide in the DNAzyme molecule. In some embodiments, the polycyclic organic moiety is attached to the 3′ terminal nucleotide of the DNAzyme. In some embodiments, the polycyclic organic moiety is attached to the 5′ terminal nucleotide of the DNAzyme. In some embodiments, the polycyclic organic moiety is attached to an internal nucleotide. In some embodiments, the polycyclic organic moiety is attached at a 2′ position of a ribose group of a nucleotide. In some embodiments, the polycyclic organic moiety is attached to another position of a nucleotide. In some embodiments, the polycyclic organic moiety can be attached to any nucleotide within the DNAzyme molecule, as long as the silencing activity (catalytic activity) of the DNAzyme is not compromised.

### Linkers

As described throughout for DNAzyme conjugates, in some embodiments, a linker is used to conjugate a DNAzyme to the polycyclic organic moiety having 3-5 rings. In some embodiments, the DNAzyme is directly conjugated to the polycyclic organic moiety.

As described throughout for DNAzyme conjugates, in some embodiments, a linker is used to conjugate a DNAzyme to the penetration-enhancing moiety (which, as described herein, may comprise a polycyclic ring system and an alkyl moiety). In some embodiments, the DNAzyme is directly conjugated to the penetration-enhancing moiety.

In some embodiments, the linker is an organic compound. In some embodiments, the organic compound is aromatic. In some embodiments, the organic compound is non-aromatic. In some embodiments, the organic compound is aliphatic.

In some embodiments, the linker is an alkyl moiety. In some embodiments, the penetration-enhancing moiety is a tocopherol, and the linker is an alkyl moiety. In some embodiments, the tocopherol is selected from alpha (α), beta (β), gamma (γ) or delta (δ) tocopherol.

In some embodiments, the polycyclic organic moiety is a steroid, and the linker is an alkyl moiety. In some embodiments, the alkyl moiety contains at least 9 carbons. In some embodiments, the alkyl is selected from the group consisting of: nonane (C₉); decane (C₁₀); undecane (C₁₁); dodecane (C₁₂); tridecane (C₁₃); tetradecane (C₁₄); pentadecane (C₁₅); hexadecane (C₁₆); heptadecane (C₁₇); octadecane (C₁₈); nonadecane (C₁₉); icosane (C₂₀); heniecosane (C₂₁); docosane (C₂₂); tricosane (C₂₃); or tetracosane (C₂₄).

In some embodiments, the linker is a polyalkylene ether. In some embodiments, the polyalkylene ether linker contains 5-15 intervening atoms between the polycyclic organic moiety and the atom bound to the DNAzyme. In some embodiments, the polyalkylene ether linker contains 5-15 intervening atoms between the ringed moiety of the penetration-enhancing moiety and the atom bound to the DNAzyme. In this context, an atom is considered bound to the DNAzyme if it binds to a phosphate moiety at an end of the DNAzyme (see Structure XV) In other embodiments, 5-14, 6-14, 7-14, 7-13, 8-13, 9-13, or 10-13, or 10-12 intervening atoms are present. In related embodiments the atom bound to the DNAzyme is selected from carbon and oxygen.

In some embodiments, the polyalkylene ether is a polyglycol. In some embodiments, the polyalkylene ether is a di- or tri-ethylene glycol. In some embodiments, the polyalkylene ether is a di- or tri-propylene glycol. In some embodiments, the polyalkylene ether is a polyalkylene glycol monoalkyl (e.g., monoethyl or monomethyl) compound. In some embodiments, the polyalkylene ether is a poly (C₂-C₃-alkylene) glycol mono (C₁-C₈-alkyl) ether having 3-15 alkylene glycol units. In some embodiments, the polyalkylene ether is selected from diethylene glycol and triethylene glycol. In some embodiments, the polyalkylene ether is a polypropylene glycol (e.g., dipropylene glycol and tripropylene glycol).

In some embodiments, the linker is a glycol. In some embodiments, the linker is a glycol polymer. In some embodiments, the glycol polymer comprises a chain of 8-20 carbons. In some embodiments, the linker is straight chained. In other embodiments, the carbon chain is branched. In some embodiments, the tocopherol is selected from alpha (α), beta (β), gamma (γ) or delta (δ) tocopherol. In some embodiments, a DNAzyme conjugate described herein comprises any of an alpha (α), beta (β), gamma (γ) or delta (δ) tocopherol. In some embodiments, the polyalkylene ether comprises Triethylene glycol (TEG). In some embodiments, the penetration-enhancing moiety is a tocopherol, and the linker is TEG. In some embodiments, the penetration-enhancing moiety is α-tocopherol linked to TEG. In some embodiments, the penetration-enhancing moiety is β-tocopherol linked to TEG. In some embodiments, the penetration-enhancing moiety is γ-tocopherol linked to TEG. In some embodiments, the penetration-enhancing moiety is δ-tocopherol linked to TEG.

In some embodiments, the linker is triethylene glycol, shown conjugated to alpha-tocopherol, to form a structure referred to as alpha-tocopherol-TEG, in Structure XV, which is further conjugated to a DNAzyme, for example but not limited to any of the DNAzymes disclosed herein. Triethylene glycol is also depicted in Structure XVI conjugated to cholesterol, to form a structure referred to as cholesterol-TEG, wherein a DNAzyme may be conjugated at the oxygen indicated by the squiggly line. Structures XV and XVI (with a DNAzyme conjugated to a structure of XVI) depict the conjugation configurations utilized in the Examples herein.

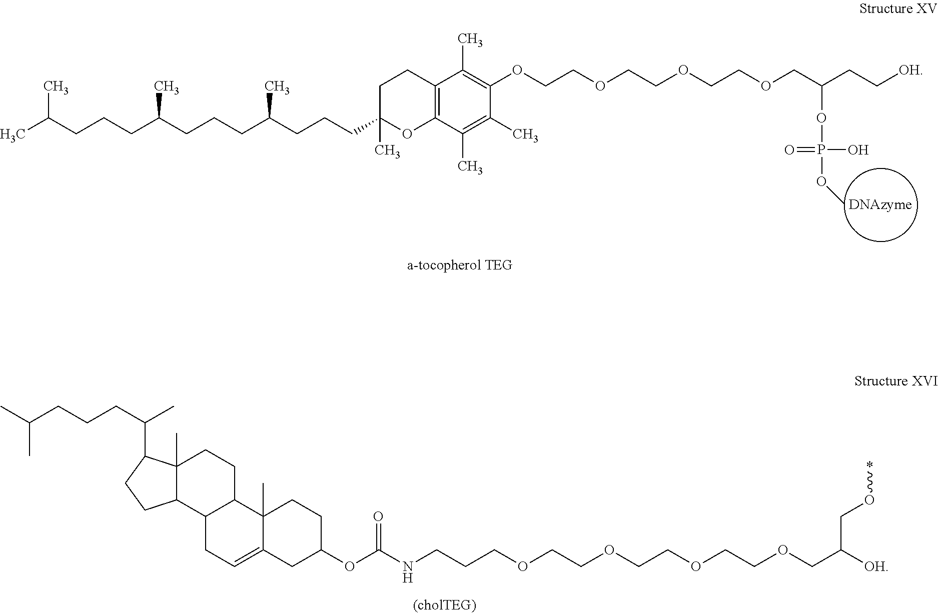 [SMILES: COP(=O)(O)OC(CCO)COCCOCCOCCOc1c(C)c(C)c2c(c1C)CC[C@@](C)(CCC[C@H](C)CCC[C@H](C)CCCC(C)C)O2 ; *OCC(O)COCCOCCOCCOCCCNC(=O)OC1CCC2(C)C(=CCC3C2CCC2(C)C(C(C)CCCC(C)C)CCC32)C1]

In some embodiments, a DNAzyme is conjugated to tocopherol using a TEG (Triethylene Glycol) linker arm. In some embodiments, a DNAzyme is conjugated to alpha-tocopherol using a TEG linker arm. In some embodiments, the tocopherol is selected from alpha (α), beta (β), gamma (γ) or delta (δ) tocopherol. In some embodiments, the DNAzyme is linked to the TEG at the 3′ of the DNAzyme. In some embodiments, the DNAzyme is linked to the TEG at the 5′ of the DNAzyme. In some embodiments, the DNAzyme comprises any DNAzyme known in the art.

In some embodiments, the linker comprises a chain of 8-20 carbons. In some embodiments, the linker is straight chained. In other embodiments, the carbon chain is branched. In some embodiments, the carbon chain is unsubstituted. In other embodiments, the carbon chain is substituted. In some embodiments, the carbons of the chain are replaced with up to 3 heteroatoms. In some embodiments, the heteroatoms are selected from nitrogen, oxygen, and sulfur; each of which represents a separate embodiment. In some embodiments, the chain is substituted with up to 3 functional groups at one or more positions, which may be any of the functional groups described herein, each of which represents a separate embodiment.

### Coupling

In some embodiments, conjugation of the DNAzyme to a polycyclic organic moiety, described herein for example but not limited to alpha-tocopherol or cholesterol, can be carried out using standard procedures in organic synthesis. In some embodiments, conjugation is via a linker attached to the polycyclic organic moiety. In some embodiments, a linker comprises TEG. In some embodiments, conjugation of the DNAzyme to a penetration-enhancing moiety (which, as described herein, may comprise a polycyclic ring system and an alkyl moiety) can be carried out using standard procedures in organic synthesis. The skilled person will appreciate that the exact steps of the synthesis will depend on the exact structure of the molecule which has to be synthesized. For instance, if the molecule is attached to the organic moiety through its 5′ end, then the synthesis is usually carried out by contacting an amino-activated oligonucleotide and a reactive activated organic moiety.

According to one embodiment, the DNAzyme is coupled to a penetration-enhancing moiety and to a protecting group (e.g., the organic moiety is coupled to the 5′ end of the DNAzyme, and the protecting group to the 3′ end, or vice versa). Use of protecting groups for coupling is well known in the art, for example see Peter G. M. Wuts, Editor. 2014 “Greene's Protective Groups in Organic Synthesis” 5th ed. Kalamazoo, Michigan, USA. John Wiley & Sons, Inc.

### DNAzymes

In some embodiments, a DNAzyme targets an RNA molecule. In some embodiments, the molecule is an RNA transcript.

In some embodiments, a DNAzyme comprises, in 5′ to 3′ order: (i) a first substrate-binding domain (also referred to herein as the “5′ arm”) comprising a sequence that base pairs with a first region of the RNA transcript; (ii) a DNAzyme catalytic core; and (iii) a second substrate-binding domain (also referred to herein as the “3′ arm”) comprising a sequence that base pairs with a second region of the RNA transcript positioned 5′ to the first region of the RNA transcript. In related embodiments, upon binding of the DNAzyme to the RNA transcript, the DNAzyme catalytic core cleaves the RNA transcript.

In some embodiments, the target molecule comprises regions that hybridize with the binding arms of the DNAzyme. In certain embodiments, the target regions are fully complementary with the binding arms of the DNAzyme. In other embodiments, the binding regions are not fully complementary with the binding arms of the DNAzyme, provided that they hybridize sufficiently with the DNAzyme such that the DNAzyme catalytic activity is not adversely affected. In some embodiments, the regions exhibit at least about 70%, 80%, 85%, 90%, or 95% complementarity. In some embodiments, the cleavage site is within a region of the substrate situated between the binding regions. In related embodiments, the terminal 5′- and 3′ ends of the cleavage site are each linked to a binding region at the appropriate corresponding terminus (e.g., 5′ to 3′) of the binding arm.

As used herein, the terms “complementarity” and “complementary” refer to a nucleic acid that can form one or more hydrogen bonds with another nucleic acid sequence by either traditional Watson-Crick or other non-traditional types of interactions. In reference to the nucleic molecules of the presently disclosed subject matter, the binding free energy for a nucleic acid molecule with its complementary sequence is sufficient to allow the relevant function of the nucleic acid to proceed, in some embodiments, binding with specificity by substrate binding domains of a DNAzyme, such that the catalytic domain of the DNAzyme is brought in to close enough proximity with a target sequence to permit catalytic cleavage of the target sequence. The degree of complementarity between the substrate binding domains of the DNAzyme and the target region of a RNA (e.g. a resistant gene mRNA) can vary, but no more than by what is required in order to permit the DNAzyme to cleave or mediate cleavage (e.g. by RNase H) of the target region. Determination of binding free energies for nucleic acid molecules to determine percent complementarity is known in the art.

As used herein, the phrase “percent complementarity” refers to the percentage of contiguous residues in a nucleic acid molecule that can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary, respectively). The terms “100% complementary”, “fully complementary”, and “perfectly complementary” indicate that all of the contiguous residues of a nucleic acid sequence can hydrogen bond with the same number of contiguous residues in a second nucleic acid sequence.

The DNAzyme additionally comprises a catalytic domain (also referred to as catalytic core) between the binding arms, generally in the form of a loop, which includes single-stranded DNA, and may optionally include double-stranded regions. The terminal 5′- and 3′ ends of the catalytic domain are each linked to a binding arm at the appropriate corresponding terminus of the binding arm. The catalytic region may incorporate modified nucleotides, including modified bases, backbone, sugars and/or linkages to the extent that such modifications do not have an adverse effect on catalytic activity (cleavage activity) of the DNAzyme.

According to one embodiment, the DNAzyme is a 10-23-type DNAzyme (i.e., comprises the 10-23 catalytic core). The term “10-23” refers to a general DNAzyme model. DNAzymes of the 10-23 model typically have a catalytic domain of 15 nucleotides, which are flanked by two substrate binding domains. The catalytic domain of 10-23 DNAzymes typically comprises the sequence ggctagctacaacga (SEQ ID NO: 28). The DNAzyme 10-23 typically cleaves mRNA strands that contain an unpaired purine-pyrimidine pair, in some cases at a position between the first and second region of the RNA transcript. The length of the substrate binding domains of 10-23 DNAzymes is variable and may be of either equal or unequal length. In some embodiments, the length of the substrate binding domains ranges between 6-14 nucleotides. In some embodiments, the length is between 8-12 nucleotides. In some embodiments, the lengths of the arms are independently selected from 7, 8, 9, 10, 11, and 12.

In some embodiments, the DNAzyme is an 8-17-type DNAzyme. In some embodiments, the DNAzyme comprises the 8-17 catalytic core. The term “8-17” refers to a general DNAzyme model. DNAzymes of the 8-17 model typically have a catalytic domain of 14 nucleotides, which are flanked by two substrate binding domains. The catalytic domain of 8-17 DNAzymes typically comprises the sequence TCCGAGCCGGACGA (SEQ ID NO: 29). The length of the substrate binding domains of 8-17 DNAzymes is variable and may be of either equal or unequal length. In some embodiments, the length of the substrate binding domains ranges between 6-14 nucleotides. In some embodiments, the length is between 8-12 nucleotides. In some embodiments, the lengths of the arms are independently selected from 7, 8, 9, 10, 11, and 12.

In certain aspects, the DNAzyme comprises a catalytic loop having the sequence: acccgguuugacuuccg (SEQ ID NO: 30). In related aspects, the DNAzyme comprises modified nucleotides. In further related aspects, modified nucleotides comprise 2′-FANA. In further related aspects, the majority of the nucleotides comprise a 2′-FANA modification. In further related aspects, all of the nucleotides comprise a 2′-FANA modification. In some embodiments, the length of the substrate binding domains ranges between 6-14 nucleotides. In some embodiments, the length is between 8-12 nucleotides. In some embodiments, the lengths of the arms are independently selected from 7, 8, 9, 10, 11, and 12.

In some embodiments, the DNAzyme molecule is selected from DNAzyme KPC-337, having a nucleotide sequence set forth in SEQ ID NO: 1; SHV-1-133 DNAzyme, set forth in SEQ ID NO: 3 or 4; In some embodiments, the DNAzyme molecule is DNAzyme TEM-588 comprising a nucleic acid sequence, set forth in SEQ ID NO: 4; USA300HOU-2333-1302 DNAzyme, set forth in SEQ ID NO: 5, 9-10, 18-19, or 23; mecA-650 DNAzyme, set forth in SEQ ID NO: 6-8, 13-15, 20 or 26; DNAzyme USA300HOU-2396-437, set forth in SEQ ID NO: 9, 10 or 23; mecA-647 DNAzyme, set forth in SEQ ID NO: 11 or 12; glpT-1122 DNAzyme, set forth in SEQ ID NO: 16 or 17; mecR1-146 DNAzyme, set forth in SEQ ID NO: 21; mecA-353 DNAzyme, set forth in SEQ ID NO: 22; USA300HOU-2333-676 DNAzyme, set forth in SEQ ID NO: 24; femA-545 DNAzyme, set forth in SEQ ID NO: 25; KPC-568 DNAzyme, set forth in SEQ ID NO: 31; KPC-36 DNAzyme, set forth in SEQ ID NO: 32; KPC-470 DNAzyme, set forth in SEQ ID NO: 33; or KPC-389 DNAzyme, set forth in SEQ ID NO: 34.

In some embodiments, the DNAzyme molecule is selected from DNAzyme KPC-563, having a nucleic acid sequence set forth in SEQ ID NO: 35. In some embodiments, the DNAzyme molecule is DNAzyme KPC-574, set forth in SEQ ID NO: 37. In some embodiments, the DNAzyme molecule is DNAzyme KPC-633, set forth in SEQ ID NO: 38. In some embodiments, the DNAzyme molecule is DNAzyme KPC-344, set forth in SEQ ID NO: 39. In some embodiments, the DNAzyme molecule is DNAzyme OXA-18-294, set forth in SEQ ID NO: 40. In some embodiments, the DNAzyme molecule is DNAzyme OXA-18-59, set forth in SEQ ID NO: 41. In some embodiments, the DNAzyme molecule is DNAzyme OXA-18-125, set forth in SEQ ID NO: 42. In some embodiments, the DNAzyme molecule is DNAzyme OXA-18-225, set forth in SEQ ID NO: 43. In some embodiments, the DNAzyme molecule is DNAzyme SHV-1-127, set forth in SEQ ID NO: 44. In some embodiments, the DNAzyme molecule is DNAzyme SHV-1-33, set forth in SEQ ID NO: 45. In some embodiments, the DNAzyme molecule is DNAzyme SHV-1-197, set forth in SEQ ID NO: 46. In some embodiments, the DNAzyme molecule is DNAzyme TEM-518, set forth in SEQ ID NO: 47. In some embodiments, the DNAzyme molecule is DNAzyme TEM-810, set forth in SEQ ID NO: 48. In some embodiments, the DNAzyme molecule is DNAzyme TEM-14, set forth in SEQ ID NO: 49.

Sequences of exemplary DNAzymes are set forth in Tables 1-2.

TABLE 1
DNAzymes effective against Klebsiella

| | | SEQ |
|---|---|---|
| | | ID |
| DNAzyme | Sequence | NO |
| KPC_337 | GCCTGTTGggctagctacaacgaCAGATATTT | 1 |
| KPC_332 | GTTGTCAGAggctagctacaacgaATTTTTCCG | 2 |
| SHV-1_133 | CCAGATCCAggctagctacaacgaTTCTATCAT | 3 |
| TEM_588 | TAGAGTAAGggctagctacaacgaAGTTCGCC | 4 |
| KPC_568 | CAGTTTTTGggctagctacaacgaAAGCTTTCC | 31 |
| KPC_36 | CATGAGAGAggctagctacaacgaAAGACAGCA | 32 |
| KPC_470 | GAACGTGGggctagctacaacgaATCGCCGA | 33 |
| KPC_389 | CGGCGTTAggctagctacaacgaCACTGTATT | 34 |
| KPC_563 | TTTTGTAAGggctagctacaacgaTTTCCGTCA | 35 |
| KPC_332 | GTTGTCAGAggctagctacaacgaATTTTTCCG | 36 |
| KPC_574 | CAGTGTCAGggctagctacaacgaTTTTGTAAG | 37 |
| KPC_633 | GTGTTTCCtccgagccggacgaTTAGCCAAT | 38 |
| KPC_344 | CCGTCATGggctagctacaacgaCTGTTGTCA | 39 |
| OXA-18_294 | ATAGACTTGggctagctacaacgaTTGTATGTG | 40 |
| OXA-18_59 | CCACGGAAggctagctacaacgaTGATTGGGA | 41 |
| OXA-18_125 | TATAAGGTAggctagctacaacgaTTCCGGTAA | 42 |
| OXA-18_225 | TTTGGCGAggctagctacaacgaTGCAAGATT | 43 |
| SHV-1_127 | CCATTTCTAggctagctacaacgaCATGCCTAC | 44 |
| SHV-1_33 | GGTGGCTAAggctagctacaacgaAGGGAGATA | 45 |
| SHV-1_197 | TTAAAGGTGggctagctacaacgaTCATCATGG | 46 |
| TEM_518 | GCTCGTCGggctagctacaacgaTTGGTATGG | 47 |
| TEM_810 | TCTATTTCGggctagctacaacgaTCATCCATA | 48 |
| TEM_14 | ACACGGAAAggctagctacaacgaGTTGAATAC | 49 |

TABLE 2
DNAzymes effective against methicillin-resistant Staphylococcus
aureus (MRSA)

| | | SEQ |
|---|---|---|
| DNAzyme | Sequence | ID NO |
| USA300HOU_ | TTTTAGTTGggctagctacaacgaGTTAGTACT | 5 |
| 2333_1302_9 nt | | |
| mecA_650_10 nt | TATTTTAGCAggctagctacaacgaAGTCATTTAA | 6 |
| mecA_661_10 nt | TGACTCATAAggctagctacaacgaTATTTTAGCA | 7 |
| mecA-658_11 nt | TGACTCATAATggctagctacaacgaTTTAGCATAGT | 8 |
| USA300HOU_ | GCTTTTTTAggctagctacaacgaTGACTAATG | 9 |
| 2396: 437_9 nt | | |
| USA300HOU_ | AGCTTTTTTAggctagctacaacgaTGACTAATGG | 10 |
| 2396: 437_10 nt | | |
| mecA_647_9 nt | TTAGCATAGggctagctacaacgaCATTTAAAT | 11 |
| mecA_647-11 nt | TTTTAGCATAGggctagctacaacgaCATTTAAATAA | 12 |
| mecA_650_9 nt | ATTTTAGCAggctagctacaacgaAGTCATTTA | 13 |
| mecA_650_11 nt | TTATTTTAGCAggctagctacaacgaAGTCATTTAAA | 14 |
| mecA_661_9 nt | GACTCATAAggctagctacaacgaTATTTTAGC | 15 |
| glpT_1122_9 nt | TTTAGTAAGggctagctacaacgaTCTTTATGG | 16 |
| glpT_1122_10 nt | ATTTAGTAAGggctagctacaacgaTTATGGGTAC | 17 |
| USA300HOU_ | TTTTAGTTGggctagctacaacgaGTTAGTACT | 18 |
| 2333_1302_9 nt | | |
| USA300HOU_ | TTTTTAGTTGggctagctacaacgaGTTAGTACTC | 19 |
| 2333_1302_10 nt | | |
| mecA-658_9 nt | TCATAATTAggctagctacaacgaTTTAGCATA | 20 |
| mecR1_146 | GAAGTCGTGggctagctacaacgaCAGATACAT | 21 |
| mecA_353 | GTTCGTTGtccgagccggacgaCGAATAATT | 22 |
| USA300HOU_ | GCTTTTTTAggctagctacaacgaTGACTAATG | 23 |
| 2396: 437 | | |
| USA300HOU_ | ATTTATTTAggctagctacaacgaAAAATTTAC | 24 |
| 2333: 676 | | |
| femA_545 | TTTTTCGTGggctagctacaacgaTTCTTTTTC | 25 |
| mecA_658_9 nt | TCATAATTAggctagctacaacgaTTTAGCATA | 26 |

In some embodiments, the sequences described herein are modified. In some embodiments, the modification comprises sequence variation of 1-6 nucleotides, in other embodiments 1-5 nucleotides, in other embodiments 1-4 nucleotides, in other embodiments 1-3 nucleotides, or in other embodiments 1-2 nucleotides, or in other embodiments 1 nucleotide. In some embodiments, the variation(s) are in the catalytic core. In some embodiments, the variation(s) are in one or both substrate binding domains (binding arms) of the DNAzyme. In some embodiments, the variation(s) are in both (a) the catalytic core and (b) one or both substrate binding domains.

In some embodiments, the described sequences are modified by deletion of 1-6 nucleotides, in other embodiments 1-5 nucleotides, in other embodiments 1-4 nucleotides, in other embodiments 1-3 nucleotides, or in other embodiments 1-2 nucleotides, or in other embodiments 1 nucleotide.

In some embodiments, the described sequences are modified by addition of 1-10 nucleotides, in other embodiments 1-8 nucleotides, in other embodiments 1-6 nucleotides, in other embodiments 1-5 nucleotides, in other embodiments 1-4 nucleotides, in other embodiments 1-3 nucleotides, or in other embodiments 1-2 nucleotides to the external ends of one or both substrate binding domains (binding arms) of the DNAzyme. In some embodiments, the additions are overhangs that are non-complementary to the target.

In some embodiments, each embodiment of sequence modification of a described sequence may be freely combined; with each combination representing a separate embodiment.

Those skilled in the art will appreciate that DNAzymes have to overcome multiple physiological barriers from the administration site to the intracellular of the bacteria. The major hurdles for internalization of DNAzymes to the bacterial cells are the cell membrane(s) and cell wall. The outer membrane of a gram-negative bacteria, which is absent in gram-positive bacteria, is a major reason for resistance to a wide range of antibiotics, due to its hydrophobic nature, which physically blocks the diffusion of some antibiotics.)

Those skilled in the art will understand that the described conjugated DNAzymes comprise nucleotides. The terms “nucleotide” “nucleobase” and the like may encompass various types of nucleotides, either deoxyribonucleotides or ribonucleotides, or analogs thereof, comprising various nucleobases. In some embodiments, a nucleotide or nucleobase includes a naturally occurring purine or pyrimidine base found in DNA (e.g., an adenine “A”, a guanine “G”, a thymine “T”, or a cytosine “C”) or a naturally occurring purine or pyrimidine base found in RNA (e.g., an adenine “A”, a guanine “G”, an uracil “U” or a cytosine “C”). In other embodiments, a nucleotide or nucleobase includes nucleic acids derived from synthetic polynucleotide and/or oligonucleotide molecules composed of naturally occurring bases, sugars, and covalent internucleoside linkages (e.g., backbone). In other embodiments, a nucleotide or nucleobase synthetic polynucleotides and/or oligonucleotides having non-naturally occurring portions, which function similarly to respective naturally occurring portions.

Various modifications to DNAzyme molecules can be made to enhance the utility of these molecules. Such modifications can enhance affinity for the nucleic acid target, increase activity, increase specificity, increase stability and decrease degradation (e.g., in the presence of nucleases), increase shelf-life, enhance half-life and/or improve resistance to nucleases.

In some embodiments, the described DNAzymes comprise one or more chemical modifications. In certain embodiments, modified bases (which may be, for example, non-naturally occurring bases) preserving the base pair specificity of the parent DNA or RNA base are considered equivalent to the DNA or RNA parent bases, e.g., a sequence mentioned herein as containing “guanine” contain instead modified forms of guanine that preserve the base pair specificity of guanine.

In some embodiments, the one or more chemical modifications are selected from the group consisting of base modifications, sugar modifications, and inter-nucleotide linkage modifications. In some embodiments, the one or more chemical modifications are selected from the group consisting of locked nucleic acids (LNA), phosphorothioate, 2-O-fluoro, 2-O-methyl, 2-O-methoxyethyl(2-MOE), 2′-O-methyl(2′-OMe), phosphoramidate morpholino, constrained ethyl (cEt), mesyl phosphoramidate, methyl phosphonate, and methyl-cytosine.

In some embodiments, the described DNAzymes comprise a 5′ end cap. In some embodiments, the 5′ end cap comprises an inverted thymidine; or bases modified with terminal amine, alkyne, azide, thiol, maleimide, or N-hydroxysuccinimide. In certain embodiments, the DNAzymes comprise a 3′ end cap. In some embodiments, the 3′ end cap comprises an inverted thymidine; or bases modified with terminal amine, alkyne, azide, thiol, maleimide, or N-hydroxysuccinimide.

In certain embodiments, the described DNAzymes comprise one or more modified sugars. In some embodiments, the described DNAzymes comprise one or more 2′ sugar substitutions (e.g., a 2′-fluoro, a 2′-amino, or a 2′-O-methyl substitution). In certain embodiments, the DNAzymes comprise locked nucleic acid (LNA), unlocked nucleic acid (UNA) and/or 2′deozy-2′fluoro-D-arabinonucleic acid (2′-FANA) sugars in their backbone.

In certain embodiments, the described DNAzymes comprise one or more (e.g., at least 1, 2, 3, 4, 5, 6, 7, 8, 9, 10 11, 12, 13, 14, 15, 16, 17, 18, 19, 20, 21, 22, 23, 24, 25, 26, 27, 28, 29, 30 31, 32, 33, 34, or 35) methylphosphonate, phosphorothioate (PS), and/or triazole internucleotide bonds, each of which is considered a separate embodiment, which may be freely combined with the other modifications described herein.

In some embodiments, the described DNAzymes comprise one or more modified bases selected from 5-(N-benzylcarboxyamide)-2′-deoxyuridine (5-BzdU), beta-naphthyl-, tryptamine, or isobutyl substituted bases; 5-methyl cytosine, or bases modified with alkyne or dibenzocyclooctyne.

In some embodiments, the modified bases comprise one or more of 5-methylcytosine (5-me-C); 5-hydroxymethyl cytosine; xanthine; hypoxanthine; 2-aminoadenine; 6-methyl and other alkyl derivatives of adenine and guanine; 2-propyl and other alkyl derivatives of adenine and guanine; 2-thiouracil, 2-thiothymine, and 2-thiocytosine; 5-halouracil and cytosine; 5-propynyl uracil and cytosine; 6-azo uracil, cytosine, and thymine; 5-uracil (pseudouracil); 4-thiouracil; 8-halo, 8-amino, 8-thiol, 8-thioalkyl, 8-hydroxyl, and other 8-substituted adenines and guanines; 5-halo, particularly 5-bromo, 5-trifluoromethyl, and other 5-substituted uracils and cytosines; 7-methylguanine and 7-methyladenine; 8-azaguanine and 8-azaadenine; 7-deazaguanine and 7-deazaadenine; and 3-deazaguanine and 3-deazaadenine. In other embodiments, the modified bases comprise one or more of 5-substituted pyrimidines, 6-azapyrimidines, and N-2, N-6, and O-6-substituted purines, including 2-aminopropyladenine, 5-propynyluracil, and 5-propynylcytosine. In other embodiments, the modified bases comprise 5-methylcytosine substitutions combined with 2′-O-methoxyethyl sugar modifications.

In certain embodiments, the described DNAzymes are DNA DNAzymes (e.g., D-DNA DNAzymes or enantiomer L-DNA DNAzymes). In other embodiments, the described DNAzymes are RNA DNAzymes (e.g., D-RNA DNAzymes or enantiomer L-RNA DNAzymes). In other embodiments, the described DNAzymes comprise a mixture of DNA and RNA.

In some embodiments, each embodiment of chemical modification of a described sequence may be freely combined; with each combination representing a separate embodiment.

In some embodiments, each embodiment of chemical or sequence modification of a described sequence may be freely combined; with each combination representing a separate embodiment.

### Target Bacteria

The term “bacteria” as used herein generally refers to a genus of prokaryotic microorganisms scientifically classified as such. Most bacteria can be classified as Gram-positive bacteria or Gram-negative bacteria.

Gram-positive bacteria relate to bacteria encapsulated by a single lipid bilayer (membrane) and a thick layer (20-80 nm) of peptidoglycan, which retains the crystal violet stain in a Gram staining technique. Exemplary Gram-positive bacteria include, but are not limited to, Actinomyces israelli, Bacillus species, Bacillus antracis, Brevibacillus, Clostridium, Clostridium perfringens, Clostridium tetani, Cornyebacterium, Corynebacterium diphtheriae, Enterococcus (e.g., Enterococcus faecium), Erysipelothrix rhusiopathiae, Lactobacillus, Listeria, Mycobacterium, Staphylococcus (e.g., Staphylococcus aureus), Streptomyces and Streptococcus.

Gram-negative bacteria relate to bacteria encapsulated by a double lipid bilayer (inner and outer cell membranes) with a relatively thin layer of peptidoglycan between the two membranes, which is unable to retain crystal violet stain in a Gram staining technique. Exemplary Gram-negative bacteria include, but are not limited to, Aerobacter, Aeromonas, Acinetobacter (e.g. Acinetobacter baumannii), Agrobacterium, Bacteroides, Bartonella, Bordetella, Borrelia, Brucella, Burkholderia, Campylobacter, Calymmatobacterium, Campylobacter, Capnocytophaga, Cardiobacterium, Citrobacter, Chlamydia, Chlamydophila, Eikenella, Enterobacter, Enterobacter aerogenes, Escherichia, Flavobacterium, Francisella, Fusobacterium, Fusobacterium nucleatum, Gardnerella, Haemophilus, Hafnia, Helicobacter, Kingella, Klebsiella (e.g. Klebsiella pneumoniae), Legionella, Leptospira, Morganella, Moraxella, Mycoplasma, Neisseria, Pasteurella (e.g. Pasteurella multocida), Plesiomonas, Prevotella, Proteus, Providencia, Pseudomonas (e.g. Pseudomonas aeruginosa), Porphyromonas, Rickettsia, Salmonella, Serratia, Shigella, Stenotrophomonas, Streptobacillus, Streptobacillus moniliformis, Stenotrophomonas, Spirillum, Treponema (e.g., Treponema pallidium, Treponema pertenue), Xanthomonas, Veillonella, Vibrio, and Yersinia.

Additional bacterial species, which are neither gram-positive or gram-negative, include, but are not limited to, Borelia.

According to one embodiment, the bacteria are pathogenic bacteria.

According to one embodiment, the bacteria cause a nosocomial infection.

According to one embodiment, the target bacteria are in a biofilm. In some embodiments, the biofilm is associated with a medical device. In some embodiments, the biofilm forms a coating on a medical device.

In some embodiments, the medical device is an implantable device. In some embodiments, the implantable device is a medical catheter. In some embodiments, the implantable device is selected from the group consisting of an orthopedic device, a surgical mesh, an artificial kidney, a cardiovascular device, a corneal implant, an ear ventilation tube, an aneurysm coil, a substitute skin graft, an intraocular lens, a vital sign monitor, a drug depot device, a neurostimulator, a silicone implant (e.g., a soft tissue silicone implant), a dental implant, a dental prosthetic, and a hernia mesh. In some embodiments, the medical device is selected from the group consisting of an embolic protection device, a ureter renal biliary stent, a urethral sling, a gastric bypass balloon, a gastric pacemaker, an insulin pump, a penile implant, an intrauterine contraceptive device, a cochlear implant, and a voice restoration device.

In some embodiments, the catheter is selected from urinary catheter, a central vascular catheter, an intravascular catheter (e.g., an arterial catheter, a central venous or PICC catheter, or a peripheral venous catheter), a peritoneal dialysis catheter, a shunt, an intubation tube (e.g., an endotracheal tube), and a gastric feeding tube (e.g., a nasogastric feeding tube).

According to one embodiment, the bacteria are resistant to an antimicrobial treatment, such as to an antibiotic. Exemplary antibiotics include, but are not limited to, beta-lactam antibiotics such as penicillin, methicillin, oxacillin, cephalosporin (e.g., third-generation oxyimino-cephalosporins e.g., ceftazidime, cefotaxime, and ceftriaxone; or methoxy-cephalosporins, e.g., cephamycin and carbapenem), cefoxitin, cefamandole, cefoperazone, imipenem, meropenem, aztreonam; macrolide antibiotics such as erythromycin, erythromycin thiocyanate; aminoglycoside antibiotics such as streptomycin, kanamycin, neomycin; tetracycline antibiotics such as minocycline, doxycycline; fluoroquinolone antibiotics such as ciprofloxacin, gemifloxacin, levofloxacin, moxifloxacin, ofloxacin; and polypeptide antibiotics such as vancomycin. According to one embodiment, the bacteria are resistant to multiple antimicrobial treatments (i.e., multidrug resistant (MDR)).

In some embodiments, the bacterium is selected from Enterococcus faecium, Staphylococcus aureus, Klebsiella pneumoniae, Acinetobacter baumannii, Pseudomonas aeruginosa, and an Enterobacter.

In some embodiments, the bacterium is Klebsiella pneumoniae (K. pneumoniae).

In some embodiments, the bacterium is Staphylococcus aureus.

In some embodiments, the bacterium is methicillin-resistant Staphylococcus aureus (MRSA).

In some embodiments, the bacterium is Pseudomonas aeruginosa.

In some embodiments, the phrase “render the bacteria susceptible to antibiotic treatment” refers to increasing susceptibility of the bacteria, such that the bacteria are more susceptible to antibiotic treatment. In some embodiments, the concentration of antibiotic required to halt bacterial growth is reduced in the presence of the conjugated DNAzyme by at least 30%, 40%, 50%, 60%, 70%, 80%, 90% or 100%, compared to bacteria treated with antibiotic alone.

In some embodiments, antibiotic treatment halts reproduction (bacteriostatic effect). In some embodiments, antibiotic treatment results in cell death (bactericidal effect). Methods for measuring reproduction or death of bacteria are well known in the art. For example, the quantity of a target bacterial species or strain in a liquid culture can be determined, for example by measuring optical density (for example at 600 nm), which correlates with the number of bacterial cells.

According to one embodiment, the target gene of a bacteria is a resistance gene.

As used herein, the term “resistance gene” or “antibiotic resistance gene” can interchangeably be used to refer to a gene conferring antibiotic resistance in bacteria.

In some embodiments, the resistance gene is a genomic gene. In some embodiments, the resistance gene is a plasmid gene. In some embodiments, the resistance gene resides on a vector that has been acquired or engineered into the bacteria.

In some embodiments, the bacterial target gene is the RNA transcript of a bacterial resistance gene.

In some embodiments, the antibiotics targeted by the resistance gene are selected from beta-lactams, macrolides, aminoglycosides, tetracyclines, fluoroquinolones and polypeptide antibiotics.

According to one embodiment, the resistance gene confers beta-lactam antibiotic resistance.

In some embodiments, the resistance gene confers carbapenem antibiotic resistance. In some embodiments, the resistance gene confers penicillin antibiotic resistance. In some embodiments, the resistance gene confers cephalosporin antibiotic resistance.

In some embodiments, the resistance gene confers monobactam antibiotic resistance.

According to one embodiment, when the bacteria are resistant to multiple antimicrobial treatments, such as in cases of mixed infection, one or more DNAzymes are used for different targets.

### Pharmaceutical Compositions

In certain embodiments, provided herein are pharmaceutical compositions comprising a conjugated DNAzyme. In some embodiments, a pharmaceutical composition comprises a therapeutically effective amount of a conjugated DNAzyme.

A skilled artisan would recognize that an “effective amount” (or, “therapeutically effective amount”) may encompass an amount sufficient to confer a beneficial or desired clinical result upon treatment. An effective amount can be administered to a subject in one or more doses. In terms of treatment, an effective amount is an amount that is sufficient to palliate, ameliorate, stabilize, reverse or slow the progression of the disease, or otherwise reduce the pathological consequences of the disease, for example but not limited to bacterial infections and sequelae thereof. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the antigen-binding fragment administered.

In some embodiments, the pharmaceutical composition comprises a plurality of conjugated DNAzymes. In some embodiments, the pharmaceutical composition comprises 2, 3, 4, 5, 6, 7, 8, 9, 10, 20, or more DNAzymes described herein. In some embodiments, the pharmaceutical composition comprising a plurality of DNAzymes, comprises equal amounts of different DNAzymes. In some embodiments, the pharmaceutical composition comprises a plurality of DNAzymes at varying ratios.

Formulation of the pharmaceutical composition may be adjusted according to applications. In particular, the pharmaceutical composition may be formulated using a method known in the art to provide rapid, continuous, or delayed release of the active ingredient after administration to mammals.

In some embodiments, the pharmaceutical composition is in a form of a sterile injectable solution.

In some embodiments, the pharmaceutical composition is suitable for administration via a route selected from the group consisting of intramuscular, subcutaneous, intravenous, intraperitoneal, inhaled, intranasal, intraarterial, intravesical, and intraocular.

In some embodiments, the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

In some embodiments, other carriers or excipients which may be used include, but are not limited to, materials derived from animal or vegetable proteins, such as the gelatins, dextrins and soy, wheat and psyllium seed proteins; gums such as acacia, guar, agar, and xanthan; polysaccharides; alginates; carboxymethylcelluloses; carrageenans; dextrans; pectins; synthetic polymers such as polyvinylpyrrolidone; polypeptide/protein or polysaccharide complexes such as gelatin-acacia complexes; sugars such as mannitol, dextrose, galactose and trehalose; cyclic sugars such as cyclodextrin; inorganic salts such as sodium phosphate, sodium chloride and aluminium silicates; and amino acids having from 2-12 carbon atoms and derivatives thereof such as, but not limited to, glycine, L-alanine, L-aspartic acid, L-glutamic acid, L-hydroxyproline, L-isoleucine, L-leucine and L-phenylalanine.

In some embodiments, solutions or suspensions used for parenteral, intradermal, or subcutaneous application include the following components: a sterile diluent such as water for injection, saline solution, fixed oils, polyethylene glycols, glycerine, propylene glycol (or other synthetic solvents), antibacterial agents (e.g., benzyl alcohol, methyl parabens), antioxidants (e.g., ascorbic acid, sodium bisulfite), chelating agents (e.g., ethylenediaminetetraacetic acid), buffers (e.g., acetates, citrates, phosphates), and agents that adjust tonicity (e.g., sodium chloride, dextrose). The pH can be adjusted with acids or bases, such as hydrochloric acid or sodium hydroxide, for example. The parenteral preparation can be enclosed in ampules, disposable syringes or multiple dose glass or plastic vials.

In some embodiments, pharmaceutical compositions adapted for parenteral administration include, but are not limited to, aqueous and non-aqueous sterile injectable solutions or suspensions, which can contain antioxidants, buffers, and solutes that render the compositions substantially isotonic with the blood of an intended recipient. Extemporaneous injection solutions and suspensions can be prepared from sterile powders, granules and tablets. Such compositions may comprise a therapeutically effective amount of a conjugated DNAzyme and/or other therapeutic agent(s), together with a suitable amount of carrier to provide the form for proper administration to the subject.

In some embodiments, the conjugated DNAzyme is encapsulated in a liposome, conjugated to a micro- or nano-particle, or embedded in a polymer matrix such as gel, PLGA, PEG, etc.

In some embodiments, lipid-based systems are used for encapsulating DNAzyme molecules for in-vivo administration to a subject, who may have a bacterial infection. Lipid-based systems include, for example, liposomes, lipoplexes and lipid nanoparticles (LNPs).

Liposomes include any synthetic (i.e., not naturally occurring) structure composed of lipid bilayers, which enclose a volume. Liposomes may be in the form of emulsions, foams, micelles, insoluble monolayers, liquid crystals, or phospholipid dispersions.

In some embodiments, for in vivo therapy, the composition of matter comprising a DNAzyme molecule is administered to the subject per se or as part of a pharmaceutical composition.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient(s)” refers to the DNAzyme molecule and, if present, the antibiotic.

Hereinafter, the phrases “physiologically acceptable carrier” and “pharmaceutically acceptable carrier”, which may be interchangeably used, refer to a carrier or a diluent that does not cause significant irritation to an organism and does not abrogate the biological activity and properties of the administered compound. An adjuvant is included under these phrases.

In some embodiments, compositions are presented in a pack or dispenser device, such as an FDA-approved kit, which may contain one or more unit dosage forms containing the active ingredient. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation formulated in a compatible pharmaceutical carrier may also be prepared, placed in an appropriate container, and labeled for treatment of an indicated condition.

According to one embodiment, there is provided an article of manufacture comprising a described conjugated DNAzyme(s), being packaged in a packaging material and identified in print, in or on the packaging material, for use in the treatment of a bacterial infection. In some embodiments, the conjugated DNAzyme may be any of the DNAzymes mentioned herein, and the use may be any of the therapeutic uses mentioned herein, which may be freely combined.

In some embodiments, there is provided a pharmaceutical composition, comprising at least one conjugated DNAzyme. In some embodiments, the composition is comprised of 1 DNAzyme, 2 DNAzymes, or more in varying ratios. In some embodiments, the conjugated DNAzyme may be any of the DNAzymes mentioned herein, and the use may be any of the therapeutic uses mentioned herein, which may be freely combined.

In some embodiments, the described therapeutic compositions may comprise, in addition to the conjugated DNAzyme(s), other known medications for the treatment of bacterial infections, e.g., antibacterial agents such as antibiotics. Exemplary antibiotics which can be used in accordance with some embodiments include, but are not limited to, penicillins (e.g., oxacillin, methicillin, amoxicillin and amoxicillin-clavulanate), monobactams (e.g., aztreonam), clavulanate acid, trimethoprim-sulfamethoxazole, cephalosporins (e.g., third-generation oxyimino-cephalosporins and methoxy-cephalosporins, including but not limited to, ceftazidime, cefotaxime, cefuroxime, ceflacor, cefprozil, loracarbef, cefindir, cefixime, cefpodoxime proxetil, ceflbuten, ceftriaxone, cephamycins (e.g., cefoxitin), carbapenems (e.g., imipenem-cilastatin, meropenem, ertapenem, doripenem, panipenem-betamipron, and biapenem)), fluoroquinolone (e.g., ofloxacin, ciprofloxacin, levofloxacin, trovafloxacin), macrolides, azalides (e.g., erythromycin, clarithromycin, and azithromycin), sulfonamides, ampicillin, tetracycline, chloramphenicol, minocycline, doxycycline, vancomycin, bacitracin, kanamycin, neomycin, gentamycin, erythromycin, spectinomycin, zeomycin, streptomycin as well as any of combinations and any derivatives thereof.

In some embodiments, the antibiotic is a beta-lactam.

In some embodiments, the antibiotic is a carbapenem.

In some embodiments, the antibiotic is a penicillin.

In some embodiments, the antibiotic is a cephalosporin.

In some embodiments, the antibiotic is a monobactam.

In some embodiments, the DNAzyme and antibiotic in any of the aforementioned compositions are packaged together.

In other embodiments, the DNAzyme and antibiotic in any of the aforementioned compositions are packaged separately.

As mentioned above, the described DNAzyme is capable of increasing susceptibility of bacteria to antibiotic therapy. Accordingly, the DNAzymes may be used alone, or with antibiotics, to treat bacterial infections.

In some embodiments, there is provided a method of treating a bacterial infection in a subject in a need thereof, comprising administering to the subject the described pharmaceutical composition.

In some embodiments, the term “subject” or “subject in need thereof” includes mammals, such as human beings, male or female, at any age which exhibits a bacterial infection.

In some embodiments, the term “subject” or “subject in need thereof” includes mammals, such as human beings, male or female, at any age which suffers from a bacterial infection.

In some embodiments, the subject is a human subject.

In some embodiments, the subject is a non-human subject (e.g., a farm animal, e.g., a mammal including e.g., a horse, a donkey, a pig, a sheep, a goat, a cow, a dog, a cat, a rabbit, a rat, a hamster, a mouse; a chicken, a duck, a goose).

According to one embodiment, the bacterial infection is caused by gram-negative bacteria.

According to one embodiment, the bacterial infection is caused by gram-positive bacteria.

According to one embodiment, the bacterial infection is caused by a single microbial species.

According to one embodiment, the bacterial infection is caused by two or more bacterial species).

According to one embodiment, the bacterial infection is caused by an antibiotic-resistant bacterial strain.

In some embodiments, the bacterial infection is caused by an Enterococcus faecium, a Staphylococcus aureus, a Klebsiella pneumoniae, an Acinetobacter baumannii, a Pseudomonas aeruginosa or an Enterobacter.

In some embodiments, the bacterial infection is caused by Klebsiella pneumoniae.

In some embodiments, the bacterial infection is caused by Staphylococcus aureus.

In some embodiments, the bacterial infection is caused by methicillin-resistant Staphylococcus aureus (MRSA).

In some embodiments, the bacterium is Pseudomonas aeruginosa.

In some embodiments, the subject has a disease or disorder selected from actinomycosis, anaplasmosis, anthrax, bacillary angiomatosis, actinomycetoma, bacterial pneumonia, bacterial vaginosis, bacterial endocarditis, bartonellosis, botulism, boutenneuse fever, brucellosis, bejel, brucellosis spondylitis, bubonic plague, Buruli ulcer, Bairnsdale ulcer, bacillary dysentery, campylobacteriosis, Carrion's disease, cellulitis, chancroid, Chlamydia infection, Chlamydia pneumonia, Chlamydia conjunctivitis, clostridial myonecrosis, cholera, Clostridium difficile colitis, diphtheria, Daintree ulcer, donavanosis, dysentery, erhlichiosis, epidemic typhus, Far East scarlet-like fever, glanders, gonorrhea, granuloma inguinale, human necrobacillosis, hemolytic-uremic syndrome, human ewingii ehrlichiosis, human monocytic ehrlichiosis, human granulocytic anaplasmosis, infant botulism, Izumi fever, Kawasaki disease, Kumusi ulder, lymphogranuloma venereum, Lemierre's syndrome, Legionellosis, leprosy, leptospirosis, listeriosis, Lyme disease, lymphogranuloma venereum, Malta fever, Mediterranean fever, myonecrosis, mycoburuli ulcer, mucocutaneous lymph node syndrome, meliodosis, meningococcal disease, murine typhus, Mycoplasma pneumonia, mycetoma, neonatal conjunctivitis, nocardiosis, Oroya fever, ophthalmia neonatorum, ornithosis, Pontiac fever, peliosis hepatis, pneumonic plague, postanginal shock including sepsis, pasteurellosis, pelvic inflammatory disease, pertussis, pneumococcal infection, pneumonia, psittacosis, parrot fever, pseudotuberculosis, Q fever, quintan fever, rabbit fever, rickettsialpox, Rocky Mountain spotted fever, Reiter syndrome, rheumatic fever, salmonellosis, scarlet fever, sepsis, septicemic plague, Searls ulcer, shigellosis, soft chancre, syphilis, streptobacillary fever, scrub typhus, trachoma, tuberculosis, tularemia, typhoid fever, typhus, tetanus, toxic shock syndrome, undulant fever, ulcus molle, Vibrio parahaemolyticus enteritis, Whitmore's disease, Waterhouse-Friderichsen syndrome, yaws, and yersiniosis.

In some embodiments, the described conjugated DNAzyme is administered to a subject in need together with an antibiotic.

In some embodiments, the antibiotic and the conjugated DNAzyme are in separate formulations.

In some embodiments, the antibiotic and the conjugated DNAzyme are in a co-formulation.

In some embodiments, a conjugated DNAzyme disclosed herein is for use in treating or preventing a bacterial infection or for use in aiding in the treatment or prevention of a bacterial infection in a subject. In some embodiments, said use increases the susceptibility of the bacteria to an antibiotic. In some embodiments, the DNAzyme is targeted to a transcript of a bacterial gene. In some embodiments, the bacterial gene is an antibiotic resistance gene. In some embodiments, the bacterial infection comprises an infection with a Gram-positive bacterium. In some embodiments, the bacterial infection comprises an infection with a Gram-negative bacterium. In some embodiments, the bacterial infection comprises an infection with a Gram-positive or a Gram-negative bacterium. In some embodiments, a subject is a human subject.

In some embodiments, disclosed herein are methods of use of a conjugated DNAzyme for treating or preventing a bacterial infection or for aiding in the treatment or prevention of a bacterial infection in a subject, said method comprising administering said DNAzyme to the subject in need. In some embodiments, said method increases the susceptibility of the bacteria to an antibiotic. In some embodiments of a method of use disclosed herein, the DNAzyme is targeted to a transcript of a bacterial gene. In some embodiments of a method of use disclosed herein, the bacterial gene is an antibiotic resistance gene. In some embodiments of a method of use disclosed herein, the bacterial infection comprises an infection with a Gram-positive bacterium. In some embodiments of a method of use disclosed herein, the bacterial infection comprises an infection with a Gram-negative bacterium. In some embodiments of a method of use disclosed herein, the bacterial infection comprises an infection with a Gram-positive or a Gram-negative bacterium. In some embodiments of a method of use disclosed herein, a subject is a human subject.

In some embodiments, a conjugated DNAzyme disclosed herein is for use increasing the susceptibility of a bacteria to an antibiotic. A skilled artisan would appreciate that use of a DNAzyme that targets a transcript of a bacterial gene, may in certain embodiments, render the bacteria susceptible to antibiotic treatment. In this way, a DNAzyme described herein aids in the treatment or prevention of a bacterial infection. In some embodiments, the DNAzyme is targeted to a transcript of a bacterial gene. In some embodiments, a conjugated DNAzyme disclosed herein is for use in treating or preventing a bacterial infection or for use in aiding in the treatment or prevention of a bacterial infection in a subject wherein the DNAzyme increases the susceptibility of the bacteria to an antibiotic. In some embodiments, disclosed herein is a method of use of a conjugated DNAzyme disclosed herein for treating or preventing a bacterial infection or for aiding in the treatment or prevention of a bacterial infection in a subject wherein the DNAzyme is administered to a subject in need, thereby increases the susceptibility of the bacteria to an antibiotic. In some embodiments, a subject being treated with a DNAzyme is also administered an antibiotic, wherein said DNAzyme increases the susceptibility of the bacteria to the antibiotic.

In some embodiments, there is a combination of a DNAzyme disclosed herein and an antibiotic, for use in treating or preventing a bacterial infection or for use in aiding in the treatment or prevention of a bacterial infection in a subject. In some embodiments, disclosed herein is a method of use of a combination comprising a DNAzyme disclosed herein and an antibiotic, for treating or preventing a bacterial infection or for aiding in the treatment or prevention of a bacterial infection in a subject.

In some embodiments of uses and methods disclosed herein, an antibiotic comprises a beta-lactam, a carbapenem, a penicillin, a cephalosporin, or a monobactam.

In some embodiments, a DNAzyme disclosed herein is for use in reducing a biofilm presence on a medical device in a subject in need thereof. In some embodiments, said use is ex vivo wherein the DNAzyme is applied to the surface of a device ex vivo. In some embodiments, a DNAzyme disclosed herein is for use in combination with an antibiotic for reducing a biofilm presence on a medical device in a subject in need thereof. In some embodiments, said use is ex vivo wherein the DNAzyme and or the antibiotic is applied to the surface of a device ex vivo. In some embodiments, a method of treating or preventing the formation of a bacterial biofilm on a surface, for example but not limited to a surface of a medical device, the method comprising applying a DNAzyme and or antibiotic to the surface of the device ex vivo. In some embodiments, use of a DNAzyme and an antibiotic is for treating or preventing the formation of a bacterial biofilm on a surface, for example but not limited to, a surface of a medical device, wherein the DNAzyme and or the antibiotic are applied to the surface. In some embodiments, use of a DNAzyme is for treating or preventing a bacterial infection ex vivo. In some embodiments, use of a DNAzyme and an antibiotic is for treating or preventing a bacterial infection ex vivo.

In some embodiments, a DNAzyme disclosed herein is for use reducing a biofilm presence on a medical device in a subject in need, said DNAzyme conjugated to a penetration enhancing moiety comprising a steroid. In some embodiments, the steroid comprises cholesterol. In some embodiments, the DNAzyme is conjugated to said steroid via a linker. In some embodiments, the linker is attached to the 3′ or 5′ terminal nucleotide of said DNAzyme.

In some embodiments, methods of use disclosed herein comprise administration of a DNAzyme disclosed herein for reducing a biofilm presence on a medical device in a subject in need, said DNAzyme conjugated to a penetration enhancing moiety comprising a steroid. In some embodiments, methods of use disclosed herein comprise use of a DNAzyme disclosed herein for reducing a biofilm presence on a medical device in a subject in need, said DNAzyme conjugated to a penetration enhancing moiety comprising a steroid, said conjugated DNAzyme applied to the device ex vivo. In some embodiments, the steroid comprises cholesterol. In some embodiments, the DNAzyme is conjugated to said steroid via a linker. In some embodiments, the linker is attached to the 3′ or 5′ terminal nucleotide of said DNAzyme.

In some embodiments, an efficient anti-bacterial treatment is determined when there is a decrease of at least about 10%, 20%, 30%, 40%, 50%, 60%, 70%, 80%, 90% or 100% or more in the number of bacterial cells or bacterial products (e.g., toxins), as compared to the number of bacterial cells or products in the subject being treated but prior to the treatment.

Throughout this application, various embodiments may be presented in a range format. It should be understood that the description in range format is merely for convenience and brevity and should not be construed as an inflexible limitation on the described scope. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate the embodiments in a non-limiting fashion.

### General Materials and Experimental Procedures

### DNAzymes

Conjugated and unconjugated DNAzymes were custom-ordered from Integrated DNA Technologies (IDT; Coralville, Iowa USA), reconstituted to 100 μM with ultrapure, DNase/RNase free water (Biological Industries, Israel), and stored at −20° C. For the conjugated DNAzymes used throughout the Examples, alpha-tocopherol or cholesterol moieties were conjugated via a triethylene glycol (TEG) linker to the 3′ end of the DNAzyme.

The tocopherol-labeled oligos were manufactured using stand solid phase oligo synthesis with commercially available Tocopherol amidites (see chemical structure of the tocopherol below).

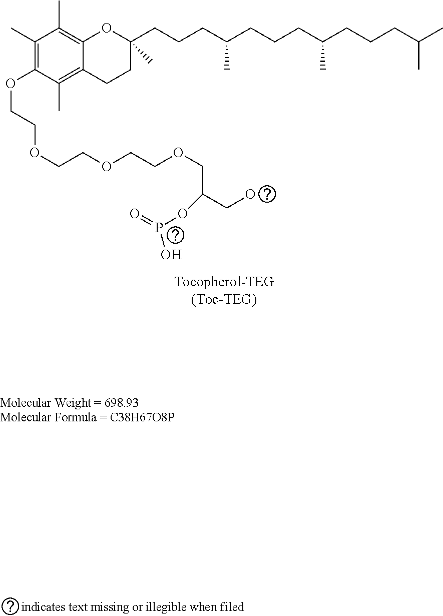

The subsequent purification of the oligos was performed using HPLC methods, suitable for this type of compound.

All methods and protocols used for manufacturing, purification, and analytical QC release tests of the compounds were generic methods known in the art. Briefly, for tocopherol-TEG coupling on 3′ terminus the protocol for standard 5′ coupling was adapted (Glen research, USA: α-Tocopherol-TEG Phosphoramidite 1-Dimethoxytrityloxy-3-O-[(9-DL-α-tocopheryl)-triethyleneglycol-1-yl]-glyceryl-2-O-[(2-cyanoethyl)-(N,N,-diisopropyl)]-phosphoramidite 1621330-39-9; or LGC Biosearch, UK: 5′-Tocopherol CE-Phosphoramidite-LK2163 or LK170) UnyLinker CPG was used for the synthesis with 3× 15 minute coupling time of a-Toc-TEG phosphoramidite+DCI as activator. The final cleavage/deprotection step was done by first 10% diethylamine treatment and then incubation in ammonium hydroxide/methylamine 1:1 (AMA) at RT (in order to avoid loss of the label).

### Synthetic Procedure:

Oligonucleotide synthesis was performed on a MerMade 48X machine using 5 umol columns prepacked with Universal CPG resin 1000 Å pore size and ˜50 μmol/g load. All amidites solutions were prepared at 0.1 M concentration in inert atmosphere using anhydrous ACN (<10 ppm) and were dried further by addition of activated molecular sieves.

Oligonucleotide synthesis was carried out by a stepwise addition of nucleotide residues to the 5′-terminus of the growing chain until the desired sequence is assembled. Each addition is referred to as a synthesis cycle (see scheme below) and consists of four chemical reactions: deblocking (detritylation), coupling, capping, oxidation.

Detritylation (de-blocking) was performed using commercial preparation of 5% Dichloroacetic acid (DCA) in toluene. The orange-colored DMT cation formed is washed out with ACN.

During the coupling step 3 to 18 equiv. of a 0.1 M amidite solution in ACN was used in 1:1 (v/v) ratio with the activator commercial solution of 0.5M 4,5-Dicyanoimidazole (DCI) solution in ACN. Upon the completion of the coupling, any unbound reagents and by-products were removed by washing with ACN.

The capping step was performed using commercial solutions of 25% acetic anhydride in ACN (CapA) and N-methylimidazole in ACN/Lutidine (20:50:30) (CapB). CapA and CapB were added in a single step in a 1:1 ratio.

The oxidation step was performed using commercial solution of 0.05 M Iodine in Pyridine/water (90:10). Sulfurization step was performed using a solution of 0.05 M Sulfurizing Reagent II (((Dimethylaminomethylidene)amino)-3H-1,2,4-dithiazoline-3-thione, DDTT) in pyridine/ACN (40:60). The oxidation step with 12 was performed after the capping step. Sulfurization step must come before capping step to minimize phosphodiester formation. All sequences were synthesized as DMT-OFF (with detritylation after final coupling). A 10% DEA in acetonitrile solution was used at the final step to remove the base labile 2-cynoethyl group (3 cycles of 5 min wash). Resin was then washed 3× with ACN and dried.

### Cleavage:

Columns were removed from the synthesis plate, mounted on the vacuum manifold and dried. Then, the support from each column was transferred into separate falcons. 1 mL of 28% NH₄OH solution and 1 mL of 40% methylamine in water were added to the support. Tubes were then sealed and incubated at RT for 2 h in thermoshaker. After that time, samples were let to cool to room temperature and supernatant is drawn from the support using syringe equipped with a needle. The solution was then filtered into a clean tube using syringe filter. Amines were then evaporated using concentrator.

Deprotected oligonucleotides solutions were analysed by UPLC-MS spectrometry and by UV spectrophotometry (Nanodrop) to quantify the target product in the crude. Deprotected oligonucleotides solutions were stored in the freezer prior to purification.

If the deprotection step was not complete, additional incubation with 28% ammonia solution for 24 h at RT was recommended.

### Bacterial Strains

Klebsiella pneumoniae (KP, ATCC® BAA-1705™) and E. coli (ATCC® BAA-2340™), both of which express beta-lactamase, were purchased from ATCC, streaked onto Luria broth (LB) agar plates (HyLabs®, Israel) and grown for 24 hours at 37° C. A single colony from each strain was arbitrarily selected and frozen in LB supplemented with 30% glycerol (HyLabs, Israel) and stored at −80° C. for all assays.

### Static Biofilm

Bacterial strains were streaked from frozen LB+glycerol stocks onto LB agar plates and incubated for 24 hours at 37° C. Single colonies were grown overnight at 37° C. in 3 ml of Mueller Hinton (MH) broth. Cultures were diluted to 0.05 OD (600 nm) in MH, then transferred to 96-well plates and incubated overnight at 37° C. without shaking. The following day, planktonic bacteria were removed, and fresh MH with treatment was added.

For biofilm formation assessment, the optical density (600 nm) of the planktonic culture was measured 20 hours after treatment, then plates were rinsed with water, and crystal violet (Sigma-Aldrich) was added to each well and incubated at room temperature for 15 minutes. Remaining crystal violet was then rinsed out with water. Absolute ethanol was added to each well and incubated at room temperature for 15 minutes. The eluate from each well was then transferred into a new 96-well plate and the optical density (600 nm) was measured.

For biofilm uptake assessment, the treatment was added for set periods of time and was then removed and replaced with PBS. The biofilm was then detached from the wells using benchtop ultrasonic cleaner (MRC), transferred to micro centrifuge tubes and analyzed using flow cytometry.

### Flow-Cell Biofilm System

Bacterial strains were streaked from frozen LB+glycerol stocks onto LB agar plates and incubated for 24 hours at 37° C. Single colonies were grown overnight at 37° C. in 3 ml of Tryptic soy broth (TSB). Cultures were diluted to 0.05 OD (600 nm) in 10% TSB, loaded into a 10 cm piece of silicon-coated latex Foley catheter (2 WAY; 16FR/CH), and incubated for 2 hours at 37° C. Catheters were then connected to a flow-cell system containing fresh 10% TSB, and fresh media was perfused through the catheter by a pump at 10 ml/hour at 37° C. After 24 hours, treatment solution was perfused into the catheter at 10 ml/hour at 37° C. for 2 hours. Bacteria were scraped from the catheter into 1 ml PBS, and decimal serial dilutions were performed. All dilutions were plated by drop-plating technique on LB agar plates. LB agar plates were incubated at 37° C., and CFU were counted the next morning.

### Flow Cytometry

Flow cytometry was performed on a ID7000™ spectral cell analyzer cytometer (Sony®). Data was analyzed using ID7000™ software. Cells were centrifuged, supernatant was aspirated, and 4% paraformaldehyde (PFA) was added for 15 minutes at 4° C. PFA was then removed, PBS was added, and samples were stained using DAPI (Biotium®). Data were collected from ˜10,000 cells per time point.

Bla-KPC Quantification from Bacterial Cells

Bacterial strains were streaked from frozen LB+glycerol stocks onto LB agar plates and incubated for 24 hours at 37° C. Single colonies were grown overnight at 37° C. in 3 ml of LB. Cultures were diluted 1:1000 in 2 ml LB+128 μg/ml meropenem, and DNAzyme was added to 1.25 μM. Cultures were incubated at 37° C. with continuous shaking for 20 min., and 1 ml culture was added to 1 ml RNAprotect® (Qiagen®). Total RNA was extracted from samples using RNeasy® Mini Kit (Qiagen®). Total RNA concentration, purity, and integrity were determined using NANODROP™ and gel electrophoresis. cDNA synthesis was performed using iScript™ cDNA synthesis (Bio-Rad®). Equal amounts of total RNA (1000 ng/20 μL) were reverse-transcribed in all samples. Reactions were incubated in a CFX96 Touch™ Real-Time PCR detection system (Bio-Rad®) by the following program: 25° C. for 5 minutes, 46° C. for 20 minutes, 95° C. for 60 seconds. Next, qPCR was performed, with primers designed using NCBI primer-BLAST and the iTaq™ Universal SYBR Green Supermix (Bio-Rad®), in a CFX96 system by the following program: 95° C. for 3 min., 39 cycles of 95° C. for 10 sec, and 55° C. for 30 sec. Melt curves were generated for each sample by heating PCR amplicons from 65-95° C. with a gradual increase of 0.5° C./0.5 s.

### Beta-Lactamase Activity Assay

Bacterial strains were streaked from frozen LB+glycerol stocks onto LB agar plates and incubated for 24 hours at 37° C. Single colonies were grown overnight at 37° C. in 3 ml of LB. Cultures were diluted 1:1000 in 2 ml LB+128 μg/ml meropenem, and DNAzyme were added to 1.25 μM. Cultures were incubated at 37° C. with continuous shaking for 1 h. Beta-lactamase activity was measured with the beta lactamase activity assay colorimetric kit from Abcam®.

### Ex Vivo Assays

Lungs were harvested from 5-10 mice (C57BL/6, 6-8 weeks old) and placed in petri dishes containing DMEM 5% FCS. The tissue was divided into circular samples 3 mm in diameter with a biopsy punch and transferred to poly-propanol tubes with 3 ml DMEM. DMEM contained meropenem and/or DNAzymes, as indicated. To each respective tube 10 μl of mid-logarithmic bacterial culture was added, with 3 technical repeats for each condition. Tubes were incubated at 37° C. for 24 hours and washed twice with PBS. For CFU of bacteria infecting ex vivo tissue culture, individual samples or their growth media were collected, samples were re-suspended in 1 ml PBS, and free-living bacteria were pelleted and re-suspended in PBS in the same volume. Cells colonizing the lung tissue were extracted by moderate sonication (2-4 pulses of 5 sec, amplitude 30% per samples). In all cases, to determine the number of viable cells, samples were serially diluted in PBS, plated on LB plates, and colonies were counted after incubation at 37° C. overnight.

### Example 1

### Design of Conjugated DNAzymes Targeting Bacterial Resistance Genes

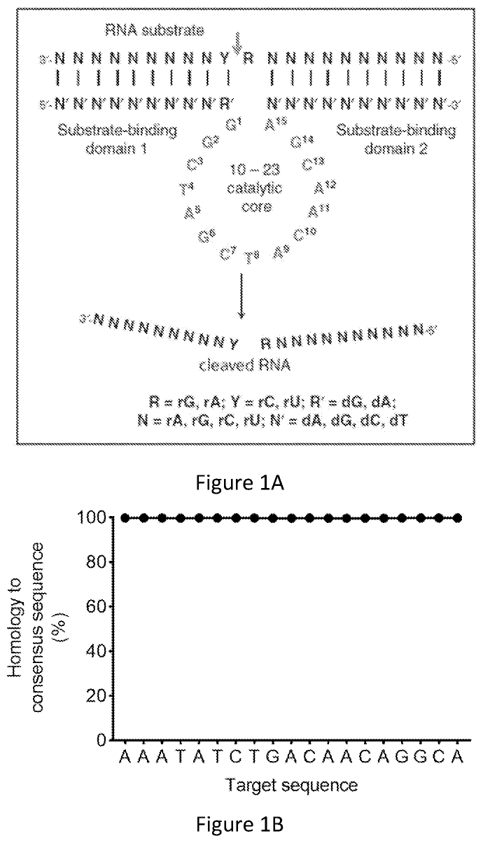

Conjugated deoxyribozymes (DNAzymes) were evaluated for ability to specifically and efficiently hydrolyze RNA transcripts of resistance genes and restore antibiotic susceptibility of resistant isolates of bacteria. The utilized DNAzymes consist of a catalytic core, flanked by two arms that recognize the DNAzyme's target RNA target through Watson-Crick base pairing and cleave RNA at a specific phosphodiester linkage (FIG. 1A).

### Example 2

### Penetration of Tocopherol-Conjugated, Cholesterol-Conjugated, and Unconjugated DNAzymes into Bacteria

The DNAzyme KPC-337 (sequence set forth in SEQ ID NO: 1) targets a conserved region in RNA transcripts of the gene bla carbapenemase of clinically relevant strains of K. pneumoniae (ec number 3.5.2.6, UniProt ID Q9F663). 30% (3845/12539) of sequenced isolates of K. pneumoniae have the annotation of bla-KPC, in which the target region (AAATATCTGACAACAGGC, bases 345-363, SEQ ID NO: 27) is conserved in 99.6% of the isolates (FIG. 1B).

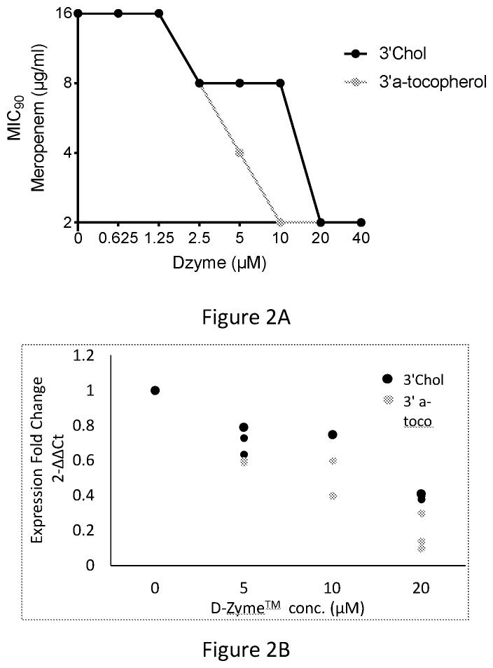

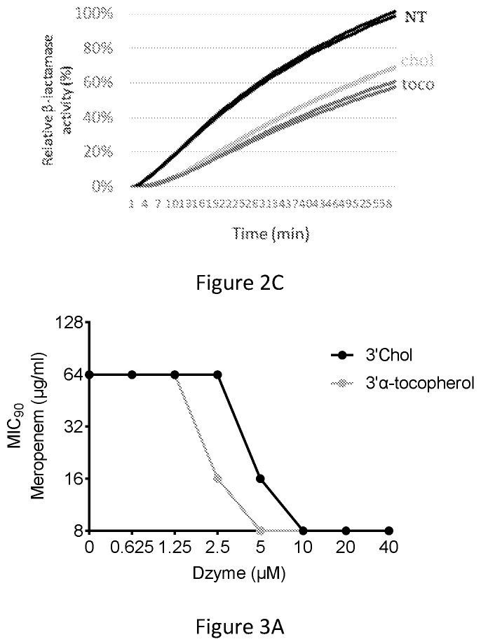

To measure penetration of conjugated DNAzymes into bacteria, DNAzymes were conjugated to alpha-tocopherol or cholesterol and administered to E. coli in an antibiotic-sensitivity test, to observe their effect on the minimum inhibitor concentration 90 (MIC₉₀; the minimum concentration required to inhibit 90% of bacterial growth) of meropenem. While both conjugated DNAzymes sharply reduced the MIC₉₀, the tocopherol-conjugated DNAzymes were more potent (FIG. 2A). Unconjugated DNAzymes did not inhibit bacterial growth.

Next, the effect of the conjugated DNAzymes on bla-KPC transcript levels in bacteria was measured. While unconjugated DNAzymes had no effect on bla-KPC transcript levels, tocopherol-conjugated and cholesterol-conjugated DNAzymes significantly reduced the transcript levels (FIG. 2B). bla-KPC knockdown relative to untreated bacteria was confirmed on the protein level by measuring secreted beta-lactamase activity using a beta-lactamase assay. Once again, the tocopherol-conjugated DNAzymes were more potent than cholesterol-conjugated DNAzymes (FIG. 2C).

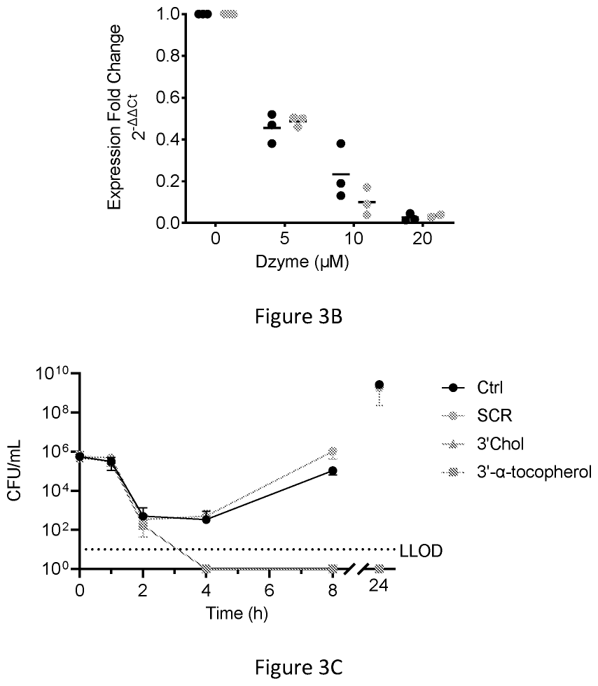

Enhanced uptake of alpha-tocopherol conjugated DNAzymes was also observed with K. pneumoniae (FIGS. 3A-B), as was potentiation of 16 mcg/mL meropenem (FIG. 3C).

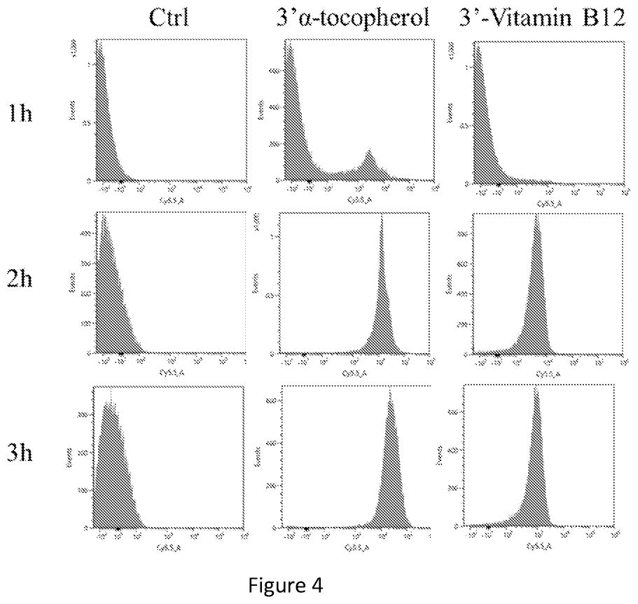

To confirm that conjugated DNAzymes successfully penetrated into bacteria, intracellular uptake was directly measured in E. coli using fluorescent DNAzymes conjugated to tocopherol or vitamin B12. Both cholesterol- and alpha-tocopherol-DNAzymes were taken up, with the tocopherol-conjugated DNAzymes taken up more rapidly, and in larger amounts per cell (FIG. 4).

### Example 3

### Penetration of Tocopherol-Conjugated, Cholesterol-Conjugated, and Unconjugated DNAzymes into Established Bacterial Biofilms

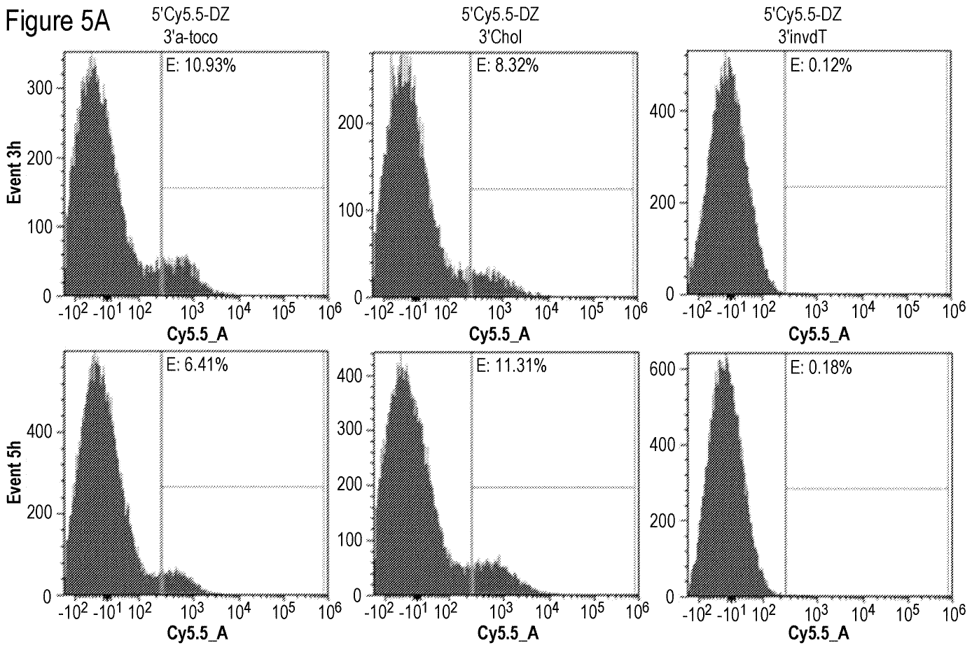

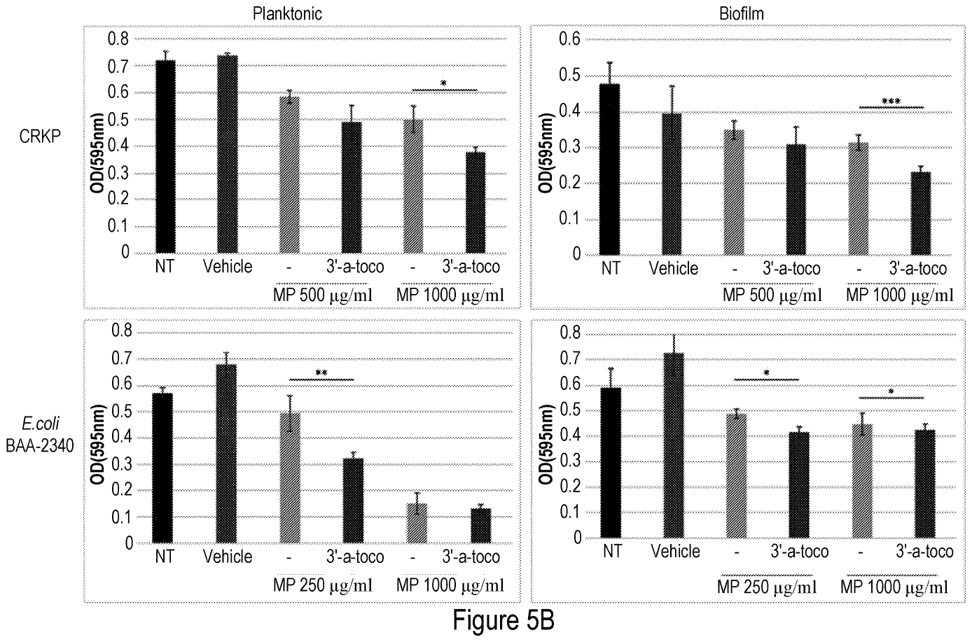

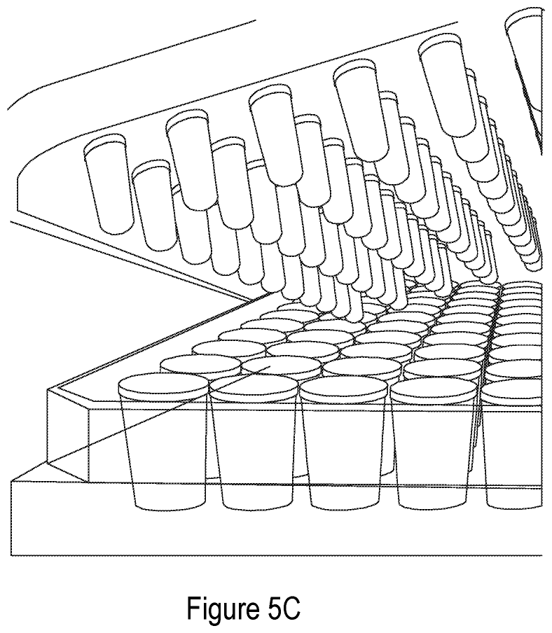

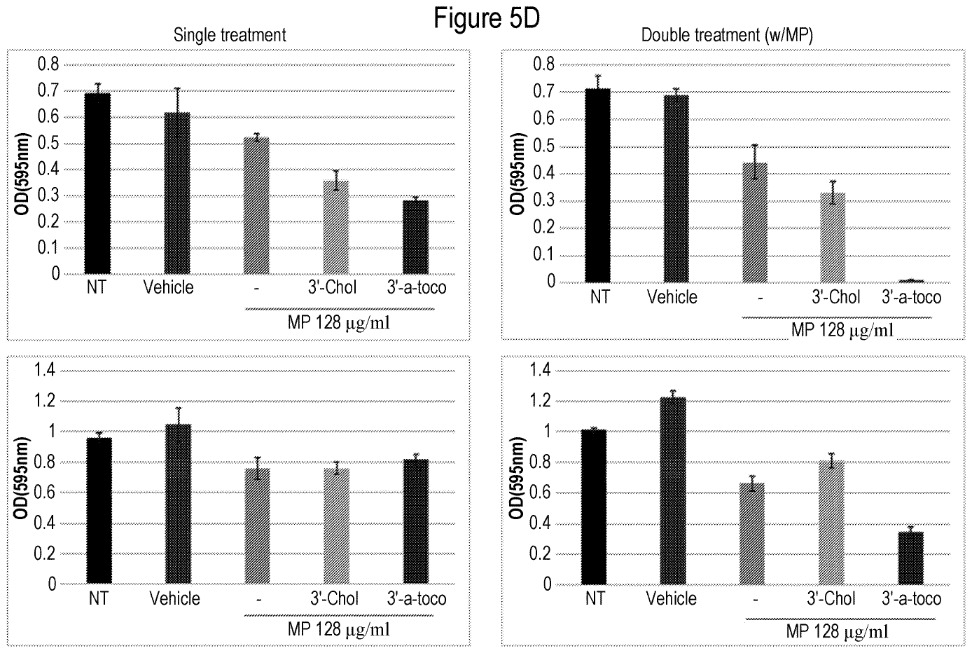

K. pneumoniae were incubated for 24 hours in 96-well plates under conditions favoring biofilm formation, and then were incubated with conjugated or unconjugated DNAzymes together with a subclinical dose (750 micrograms [mcg]/mL) of meropenem for 3 or 5 hours. While unconjugated DNAzymes were not appreciably taken up by the bacteria in the biofilm, tocopherol-conjugated and cholesterol-conjugated DNAzymes were significantly taken up (FIG. 5A).

The next experiment tested the ability of conjugated DNAzymes to inhibit biofilm growth and disperse existing biofilms. E. coli and K. pneumoniae were incubated in 96-well plates to form biofilms (as previously). After 24 hours, the growth medium and free-growing (planktonic) cells were removed, and fresh medium was added to the biofilm adhering to the dish, together with tocopherol-conjugated DNAzymes and 250 or 1000 (E. coli) 500 or 1000 (K. pneumoniae) mcg/mL meropenem. 24 hours later, planktonic bacteria and bacteria in the biofilm were measured. Tocopherol-conjugated DNAzymes reduced the amounts of both planktonic and biofilm bacteria in both species. The effect was statistically significant at least at one concentration in each case (FIG. 5B).

The next experiment tested the ability of conjugated DNAzymes to inhibit biofilm attachment and growth. K. pneumoniae were incubated in dishes to form biofilms, in wells covered by a lid that jutted into the culture and itself served as a substrate for biofilm formation (FIG. 5C). After 24 hours, the lid was removed and placed into a fresh well containing tocopherol-conjugated or cholesterol-conjugated DNAzymes and 128 mcg/mL meropenem. In some samples, a second treatment of antibiotic alone was administered 2 hours after the first treatment. 24 hours later, amounts of planktonic and biofilm bacteria (top and bottom rows of FIG. 5D) were measured. A single DNAzyme+antibiotic treatment reduced planktonic bacteria but not new biofilm formation. Two treatments reduced all tested parameters, while the tocopherol-conjugated DNAzymes being particularly potent (FIG. 5D).

### Example 4

### Conjugated DNAzyme Reverse Catheter-Associated Biofilm Formation

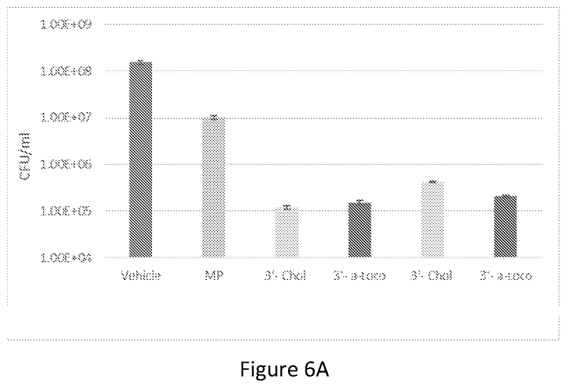

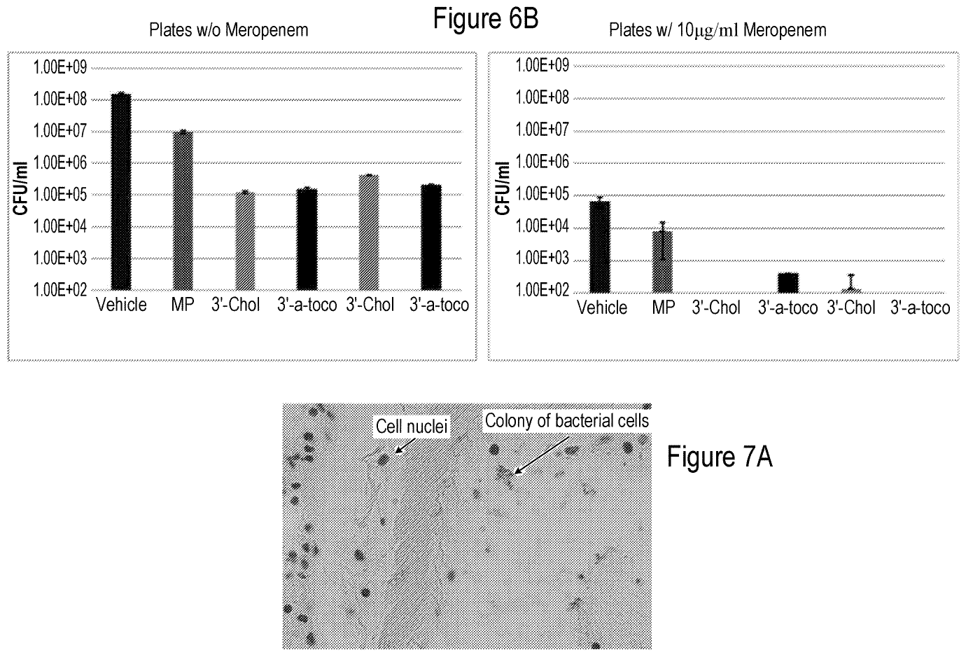

To test the effect of conjugated DNAzymes on established, catheter-associated biofilms, K. pneumoniae were incubated in catheters for 2 hours in static conditions and then for 24 hours with fresh media flow, after which they were subject to a 2-hour perfusion with 750 or 1000 mcg/mL meropenem, alone or in combination with tocopherol-conjugated or cholesterol-conjugated DNAzymes. After the incubation, the tubing was cut open and bacteria were scraped from the inside of the tubing, after which the CFU were determined by replating and observing colony formation. Conjugated DNAzymes+antibiotic, but not antibiotic alone, significantly disrupted the established biofilms (FIG. 6A). When CFU formation was tested in plates containing a much lower concentration (10 mcg/mL) of meropenem, the prior treatment with conjugated DNAzymes+meropenem nearly completely abolished colony formation (FIG. 6B), showing that antibiotic resistance was largely, if not entirely, abrogated in the disrupted biofilms.

### Example 5

### Ex Vivo Effect of a DNAzyme Targeting KPC Gene in Klebsiella pneumoniae

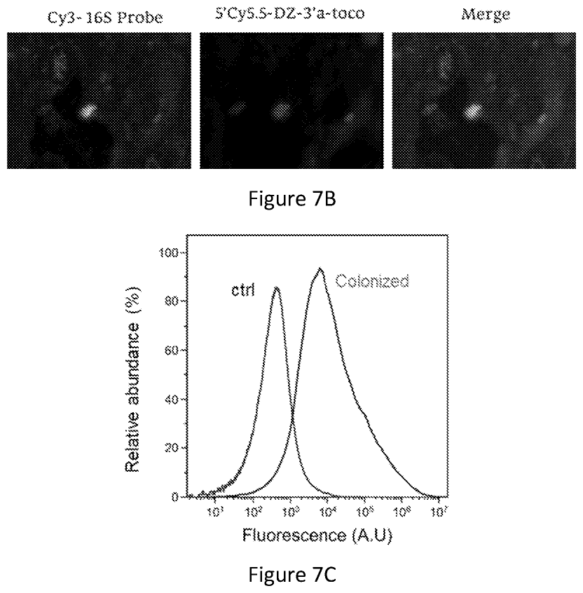

A lung ex-vivo infection model was used to test uptake of conjugated DNAzymes during colonization by K. pneumoniae. Gram staining confirmed colonization of the lung tissue samples (FIG. 7A). Internalization of tocopherol-conjugated DNAzymes was shown by colocalization of fluorescent DNAzymes and bacterial colonies (FIG. 7B) and flow cytometry of bacteria isolated from the infected tissue (FIG. 7C).

Although the conjugated DNAzymes described herein have been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant that citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A DNAzyme conjugated to a penetration-enhancing moiety, wherein said penetration-enhancing moiety comprises a benzodihydropyran ring system and an alkyl moiety, and wherein said DNAzyme is conjugated to said benzodihydropyran ring system.

2. The DNAzyme according to claim 1, wherein said benzodihydropyran ring system comprises a chromane moiety.

3. The DNAzyme according to claim 1, wherein said alkyl moiety comprises a chain of 8-20 carbons.

4. The DNAzyme according to claim 1, wherein said penetration-enhancing moiety comprises a tocopherol.

5. The DNAzyme according to claim 1, wherein said penetration-enhancing moiety comprises alpha-tocopherol.

6. The DNAzyme according to claim 1, further comprising a linker conjugating the DNAzyme to the benzodihydropyran ring system.

7. The DNAzyme according to claim 1, wherein said benzodihydropyran ring system or said linker is attached to the 3′ terminal nucleotide of said DNAzyme; or wherein said benzodihydropyran ring system or said linker is attached to the 5′ terminal nucleotide of said DNAzyme.

8. (canceled)

9. The DNAzyme according to claim 6, wherein said linker comprises Triethylene glycol (TEG).

10. The DNAzyme of claim 1, wherein said DNAzyme is a 10-23-type DNAzyme molecule.

11. A method of treating or preventing a bacterial infection in a subject in need thereof or of aiding in the treatment or prevention of a bacterial infection in a subject in need thereof, said method comprising administering to the subject a DNAzyme conjugated to a penetration-enhancing moiety, wherein said penetration-enhancing moiety comprises a benzodihydropyran ring system and an alkyl moiety, and wherein said DNAzyme is conjugated to said benzodihydropyran ring system, thereby treating or preventing the bacterial infection in the subject.

12. The method according to claim 11, wherein said DNAzyme increases the susceptibility of a bacterium to an antibiotic, wherein optionally said antibiotic comprises a beta-lactam, a carbapenem, a penicillin, a cephalosporin, or a monobactam.

13. The method according to claim 11, wherein said DNAzyme is targeted to a transcript of a bacterial gene.

14. The method according to claim 13, wherein said bacterial gene is an antibiotic resistance gene.

15. The method according to claim 11, wherein said bacterial infection is an infection with a Gram-positive bacterium, or said bacterial infection is an infection with a Gram-negative bacterium.

16. (canceled)

17. The method according to claim 11, wherein said subject is a human subject.

18. The method according to claim 11, further comprising administering an antibiotic to said subject in need.

19. The method according to claim 18, wherein said antibiotic comprises a beta-lactam, a carbapenem, a penicillin, a cephalosporin, or a monobactam.

20. The method according to claim 11, wherein said infection is associated with a biofilm presence on a medical device and said method reduces the biofilm presence on the medical device in the subject.

21. A method of reducing a biofilm presence on a medical devise in a subject in need thereof, said method comprising administering a DNAzyme conjugated to a penetration enhancing moiety comprising a steroid, wherein optionally said steroid comprises cholesterol.

22. (canceled)

23. The method according to claim 21, wherein said DNAzyme is conjugated to said steroid via a linker, wherein optionally said linker is attached to the 3′ or 5′ terminal nucleotide of said DNAzyme.

24. (canceled)
